(19) United States
(12) Patent Application Publication
(10) Pub. No.: US 2024/0199656 A1
(43) Pub. Date: Jun. 20, 2024

(54) BORON NEUTRON CAPTURE THERAPY (BNCT) PROBE

(71) Applicant: The University of Tokyo, Tokyo (JP)

(72) Inventors: Yasuteru URANO, Kanagawa (JP); Mako KAMIYA, Kanagawa (JP); Junya TSUNETOMI, Tokyo (JP)

(73) Assignee: The University of Tokyo, Tokyo (JP)

(21) Appl. No.: 18/277,623

(22) PCT Filed: Feb. 21, 2022
(86) PCT No.: PCT/JP2022/006876

§ 371 Date: Nov. 13, 2023

Related U.S. Application Data
(60) Provisional application No. 63/151,107, filed on Feb. 19, 2021.

(51) Int. Cl. C07F 5/02; A61K 51/04; A61N 5/10

(52) U.S. Cl. CPC C07F 5/027; A61K 51/04; A61N 5/1077; A61N 2005/1098

(57) ABSTRACT

[Problem] To provide a novel compound promising as a probe for a boron neutron capture therapy (BNCT).

[Solution] A compound represented by general formula (I) below or a salt thereof.

### TECHNICAL FIELD

The present invention relates to a novel compound that is promising as a boron neutron capture therapy (BNCT) probe, and a pharmaceutical composition used in a boron neutron capture therapy using the compound.

### BACKGROUND ART

It is known that ¹⁰B, which is one of boron isotopes, emits Li nuclei and α-rays when capturing neutrons. A treatment method utilizing the property of ¹⁰B is a boron neutron capture therapy (BNCT). In the BNCT, first, ¹⁰B is accumulated in cancer cells, and a neutron capture reaction is induced by irradiation with neutron rays (see FIG. 1).

Specifically, in the BNCT, ¹⁰B generates α-rays and Li nuclei by a reaction with neutrons, and both of them have a high linear energy transfer (LET), and therefore have a higher killing ability than that of X-rays used in a conventional radiation therapy. In addition, α-rays whose range distance is 5 to 9 μm, which is shorter than a diameter of a single cell, kill only cancer cells and do not damage adjacent normal cells. Therefore, when ¹⁰B can be delivered only to cancer cells, it is theoretically possible to perform a cancer-selective radiation treatment at a single cell resolution level. In addition, since the BNCT has both high killing ability and cancer cell selectivity, it can also be used in cancer types having high malignancy such as radiation resistance.

In the BNCT, in order to efficiently emit α-rays at a cancer site and suppress damage to a normal site, two of (1) accumulation of ¹⁰B at a high concentration (˜ several mM) and (2) high tumor selectivity are significantly important.

Although only two types of drugs, BSH and BPA (p-boronophenylalanine), are currently used in clinical studies, it is difficult to accumulate a high concentration of ¹⁰B in cancer cells with high selectivity and maintain the high concentration for a sufficient time with both drugs, and thus it is not possible to sufficiently derive an excellent therapeutic effect of the BNCT. That is, in order to complete the BNCT as an ideal cancer treatment technology that minimizes damage to normal tissues, it is required to develop a completely novel BNCT drug that realizes a high concentration and tumor-selective retention of ¹⁰B.

### SUMMARY OF INVENTION

### Technical Problem

An object of the present invention is to provide a novel compound that is promising as a probe for a boron neutron capture therapy (BNCT).

### Solution to Problem

SPIDER-βgal that is a fluorescent probe developed by the laboratory of the present inventors reacts with β-galactosidase to generate a quinone methide intermediate, and the quinone methide intermediate is tagged with various nucleophiles in the cell, such that only β-galactosidase-expressing cells can be fluorescently labeled with resolution at a single cell level.

Furthermore, in the laboratory of the present inventors, a cancer-selective prodrug type anticancer agent was developed using quinone methide chemistry. It is suggested that the anticancer agent disturbs intracellular redox balance by utilizing high reactivity of an azaquinone methide intermediate to be generated and consuming intracellular nucleophiles, and induces cancer cells to apoptosis (WO 2019/172210 A and the like).

Based on these facts, the present inventors have considered that when quinone methide chemistry is adopted and covalent bond formation with an intracellular nucleophile can be utilized as an intracellular retention mechanism, it is possible to develop a more robust intracellular retention type BNCT drug, and it is possible to solve the problem of persistence of the boron concentration of the existing BNCT drug, thereby completing the present invention.

That is, the present invention provides the following.
[1] A compound represented by the following General Formula (I) or a salt thereof,

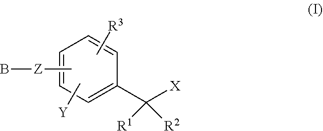

in the formula,

X is selected from the group consisting of a fluorine atom, an ester group (—OC(═O)—R′), a carbonate group (—OCO₂—R′), a carbamate group (—OCONH—R′), phosphoric acid and an ester group thereof (—OP(═O) (—OR′) (—OR″)), and sulfuric acid and an ester group thereof (—OSO₂—OR′),

where R′ and R″ are each independently selected from a substituted or unsubstituted alkyl group or a substituted or unsubstituted aryl group;

Y is —NH—CO—L, —NH-L′, or —OL′,

where L is a partial structure of an amino acid,

L′ is a saccharide or a partial structure of a saccharide, a saccharide having a self-cleaving linker, or an amino acid or a peptide having a self-cleaving linker;

R¹ and R² are each independently selected from a hydrogen atom or a monovalent substituent;

R³ is a hydrogen atom or one to three identical or different monovalent substituents present on a benzene ring;

Z represents a single bond or a linking group; and

B represents a group containing ¹⁰B.
[2] The compound or a salt thereof according to [1], wherein B is a group derived from a compound having at least one boron atom in a molecule.
[3] The compound or a salt thereof according to [1] or [2], wherein B is a group derived from a boron cluster.
[4] The compound or a salt thereof according to [3], wherein the boron cluster has a polyhedral structure.
[5] The compound or a salt thereof according to any one of [1] to [4], wherein B is a group derived from closo-dodecaborate, closo-carborane, nidocarborane, a bisdicarbolide metal complex, GB10, 1,2-dicarbacloso-dodecarborane, 1,7-dicarba-closo-dodecarborane, 1,12-dicarba-closo-dodecarborane, dicarba-closo-decarborane, or sulfur-substituted undecahydrododecaborate.
[6] The compound or a salt thereof according to any one of [1] to [5], wherein the linking group is selected from the group consisting of an alkylene group (where one or more —CH₂— of the alkylene group may be substituted with —O—, —S—, —NH—, or —CO—), arylene (including heteroarylene), cycloalkylene, an alkoxyl group, a polyethylene glycol chain, and a group constituted by optionally binding two or more groups selected from these groups.
[7] The compound or a salt thereof according to any one of [1] to [6], wherein the partial structure of the amino acid of L constitutes an amino acid, an amino acid residue, a peptide, or a part of an amino acid together with C═O to which L is bonded.
[8] The compound or a salt thereof according to any one of [1] to [6], wherein the partial structure of the saccharide of L′ constitutes a saccharide or a part of a saccharide together with O to which L′ is bonded.
[9] The compound or a salt thereof according to any one of [1] to [8], wherein -Y in General Formula (I) is bonded to —(R¹) (R²)X at an ortho position or a para position of a benzene ring.
[10] The compound or a salt thereof according to any one of [1] to [9], wherein Y has a structure selected from the following.

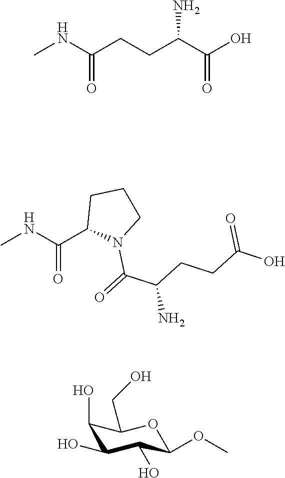

[11] The compound or a salt thereof according to any one of [1] to [10], wherein X is a fluorine atom or an ester group (—OC (═O)—R′).
[12] The compound or a salt thereof according to any one of [1] to [11], wherein RI and R² are each independently selected from a hydrogen atom or a fluorine atom.
[13] The compound or a salt thereof according to any one of [1] to [12], wherein the monovalent substituent of R³ is selected from the group consisting of an alkyl group, an alkoxycarbonyl group (—C(═O)—OR′), a nitro group, an amino group, a hydroxyl group, an alkylamino group (—NHR′ or —NR′₂), an alkoxy group (—OR′), an ester group (—O—CO—R′), an amide group (—NHCOR′), a halogen atom, a boryl group, and a cyano group, where R′ is a substituted or unsubstituted alkyl group or a substituted or unsubstituted aryl group, and when there are two or more R's, R's may be the same as or different from each other.
[14] The compound or a salt thereof according to

[13], wherein the monovalent substituent of R³ is an alkyl group or an alkoxy group.
[15] The compound or a salt thereof according to

[13], wherein the monovalent substituent of R³ is a halogen atom.
[16] The compound or a salt thereof according to any one of to [15], wherein one or more of the monovalent substituents of R³ are alkyl groups or alkoxy groups, and one or more of the monovalent substituents of R³ are halogen atoms.
[17] The compound or a salt thereof according to any one of [1] to [12], wherein all of R's are hydrogen atoms.
[18] A pharmaceutical composition comprising the compound according to any one of [1] to or a pharmaceutically acceptable salt thereof.
[19] The pharmaceutical composition according to [18], wherein the pharmaceutical composition is used in a boron neutron capture therapy.
[20] The pharmaceutical composition according to [19], wherein the pharmaceutical composition is accumulated in cancer cells by acting selectively on cells by a cancer cell-specific enzyme activity.
[21] The pharmaceutical composition according to [20], wherein the enzyme is a peptidase or a glycosidase.
[22] A method for diagnosing, treating, or diagnosing and treating a disease or a symptom that may cause the disease, the method including:
(A) administering, to a subject having or suspected of having a disease or a symptom, a pharmaceutical composition containing the compound according to any one of claims 1 to 17 or a pharmaceutically acceptable salt thereof; and
(B) irradiating a ¹⁰B atom localized in a target tissue of the subject with a neutron beam to perform a boron neutron capture therapy on the target tissue.

### Advantageous Effects of Invention

According to the present invention, it is possible to provide a novel compound that is promising as a probe for a boron neutron capture therapy (BNCT).

### BRIEF DESCRIPTION OF DRAWINGS

FIG. 1 is a schematic view of a boron neutron capture therapy (BNCT).

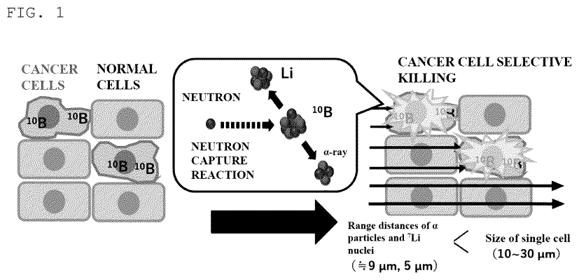

FIG. 2 illustrates a schematic view of a mechanism by which a compound of the present invention is retained in a cell through a reaction with a cancer biomarker enzyme.

FIG. 3 illustrates the results of confirming reactivity of gGlu-4OCB-FMA with a purified enzyme.

FIG. 4 illustrates the results of confirming reactivity of EP-4OCB-FMA with a purified enzyme.

FIG. 5 illustrates the results of confirming reactivity of EP-4OCB-MA with a purified enzyme.

FIG. 6 illustrates the results of measuring cell viability in an EP-4OCB-FMA treatment for 24 hours in the presence or absence of CCK8 and sitagliptin.

FIG. 7 illustrates the results of measuring cell viability in an EP-4OCB-FMA treatment for 3 hours or 24 hours in the presence or absence of CCK8 and sitagliptin.

FIG. 8 illustrates the results of measuring cell viability in an EP-4OCB-MA treatment for 24 hours in the presence or absence of CCK8 and sitagliptin, in which a mean value±a standard deviation of concentrations compared to an untreated case is illustrated (n=3 biological replicates).

FIG. 9 illustrates a protocol used in a test of Example 4.

FIG. 10 illustrates the results of quantification of an extracellular boron concentration in Example 4.

FIG. 11 illustrates the results of quantification of an intracellular boron concentration in Example 4.

FIG. 12 illustrates the results of evaluating reactivity of an azaquinone methide intermediate with a nucleophilic group for EP-4OCB-FMA.

### DESCRIPTION OF EMBODIMENTS

In the present specification, the “halogen atom” means a fluorine atom, a chlorine atom, a bromine atom, or an iodine atom.

In the present specification, the “alkyl” may be any of linear alkyl, branched alkyl, cyclic alkyl, or an aliphatic hydrocarbon group composed of a combination thereof. The number of carbon atoms in the alkyl group is not particularly limited, and is, for example, 1 to 6 carbon atoms (C₁-6), 1 to 10 carbon atoms (C₁-10), 1 to 15 carbon atoms (C₁-15), or 1 to 20 carbon atoms (C₁-20). In a case where the number of carbon atoms is specified, it means “alkyl” having the number of carbon atoms in the range of the number. For example, C₁-8 alkyl includes methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. In the present specification, the alkyl group may have one or more arbitrary substituents. Examples of such a substituent include an alkoxy group, a halogen atom, an amino group, a mono- or disubstituted amino group, a substituted silyl group, and acyl, but are not limited thereto. In a case where the alkyl group has two or more substituents, these substituents may be the same as or different from each other. The same applies to alkyl moieties of other substituents including alkyl moieties (for example, an alkoxy group, an arylalkyl group, and the like).

In the present specification, in a case where a certain functional group is defined as “which may be substituted”, the type of substituent, a substitution position, and the number of substituents are not particularly limited, and in a case where a certain functional group has two or more substituents, these substituents may be the same as or different from each other. Examples of the substituent include an alkyl group, an alkoxy group, a hydroxyl group, a carboxyl group, a halogen atom, a sulfo group, an amino group, an alkoxycarbonyl group, and an oxo group, but are not limited thereto. Substituents may be further present in these substituents. Examples thereof include a halogenated alkyl group and a dialkylamino group, but are not limited thereto.

In the present specification, the “aryl” may be either a monocyclic or fused polycyclic aromatic hydrocarbon group, and may be an aromatic heterocyclic ring containing one or more heteroatoms (for example, an oxygen atom, a nitrogen atom, a sulfur atom, or the like) as ring-constituting atoms. In this case, it may also be referred to as “heteroaryl” or “heteroaromatic”. Even in a case where aryl is a single ring or a fused ring, it may be bonded at all possible positions. Non-limiting examples of the monocyclic aryl include a phenyl group (Ph), a thienyl group (2- or 3-thienyl group), a pyridyl group, a furyl group, a thiazolyl group, an oxazolyl group, a pyrazolyl group, a 2-pyrazinyl group, a pyrimidinyl group, a pyrrolyl group, an imidazolyl group, a pyridazinyl group, a 3-isothiazolyl group, a 3-isoxazolyl group, a 1,2,4-oxadiazol-5-yl group, and a 1,2,4-oxadiazol-3-yl group.

Non-limiting examples of the fused polycyclic aryl include a 1-naphthyl group, a 2-naphthyl group, a 1-indenyl group, a 2-indenyl group, a 2,3-dihydroinden-1-yl group, a 2,3-dihydroinden-2-yl group, a 2-anthryl group, an indazolyl group, a quinolyl group, an isoquinolyl group, a 1,2-dihydroisoquinolyl group, a 1,2,3,4-tetrahydroisoquinolyl group, an indolyl group, an isoindolyl group, a phthalazinyl group, a quinoxalinyl group, a benzofuranyl group, a 2,3-dihydrobenzofuran-1-yl group, a 2,3-dihydrobenzofuran-2-yl group, a 2,3-dihydrobenzothiophen-1-yl group, a 2,3-dihydrobenzothiophen-2-yl group, a benzothiazolyl group, a benzimidazolyl group, a fluorenyl group, and a thioxanthenyl group. In the present specification, the aryl group may have one or more arbitrary substituents on its ring. Examples of the substituent include an alkoxy group, a halogen atom, an amino group, a mono- or disubstituted amino group, a substituted silyl group, and acyl, but are not limited thereto. In a case where the aryl group has two or more substituents, these substituents may be the same as or different from each other. The same applies to aryl moieties of other substituents including aryl moieties (for example, an aryloxy group, an arylalkyl group, and the like).

In the present specification, the “alkoxy group” is a structure in which the alkyl group is bonded to an oxygen atom, and examples thereof include a linear alkoxy group, a branched alkoxy group, a cyclic alkoxy group, and a saturated alkoxy group composed of a combination thereof. Preferred examples thereof include a methoxy group, an ethoxy group, an n-propoxy group, an isopropoxy group, a cyclopropoxy group, an n-butoxy group, an isobutoxy group, an s-butoxy group, a t-butoxy group, a cyclobutoxy group, a cyclopropylmethoxy group, an n-pentyloxy group, a cyclopentyloxy group, a cyclopropylethyloxy group, a cyclobutylmethyloxy group, an n-hexyloxy group, a cyclohexyloxy group, a cyclopropylpropyloxy group, a cyclobutylethyloxy group, and a cyclopentylmethyloxy group.

In the present specification, the “alkylene” is a divalent group composed of a linear or branched saturated hydrocarbon, and examples thereof include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, and 2,2-di-n-propyltrimethylene.

### 1. Compound Represented by General Formula (I) or Salt Thereof.

One embodiment of the present invention is a compound represented by the following General Formula (I) or a salt thereof (hereinafter, also referred to as a “compound of the present invention”).

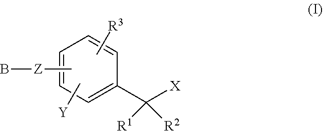

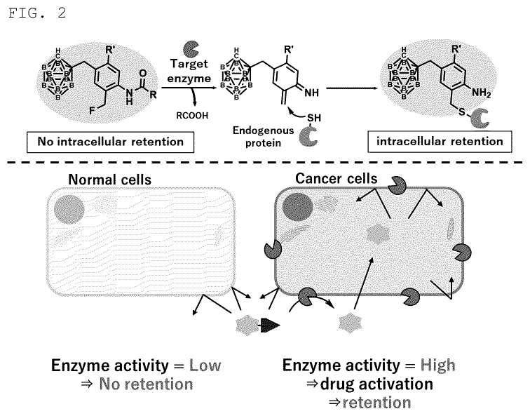

Although not intended to be bound by theory, in the present invention, the present inventors have considered that it is possible to target a cancer biomarker enzyme, incorporate a substrate site thereof into a drug molecule, expose a quinone methide intermediate only after the substrate site is cleaved by an enzymatic reaction, and tag the quinone methide intermediate with an intracellular nucleophile, thereby allowing the compound to be retained in a cell, and have made a molecular design of the compound. As a result, it was found that the compound represented by General Formula (I) can maintain a high boron concentration selectively and continuously in cancer cells, and is useful as a novel probe for a BNCT. FIG. 2 illustrates a schematic view of a mechanism by which a compound of the present invention is retained in a cell through a reaction with a cancer biomarker enzyme.

In General Formula (I), Y is an enzyme recognition site, and is a site at which a part thereof is cleaved by a cancer cell-specific enzyme activity to induce formation of a quinone methide.

Y can be selected according to the type of the target enzyme. In a case where the cancer biomarker enzyme as the target enzyme is a glycosidase, Y is selected from a group derived from a saccharide, and in a case where the target enzyme is a peptidase, Y is selected from a group derived from an amino acid and a group containing an amino acid.

In General Formula (I), Y is preferably —NH—CO-L, —NH-L′, or —OL′.

Here, L is a partial structure of an amino acid. The partial structure of the amino acid of L means that it constitutes an amino acid, an amino acid residue, a peptide, or a part of an amino acid together with C═O to which L is bonded.

In the present specification, as the “amino acid”, any compound can be used as long as it is a compound having both an amino group and a carboxyl group, and includes natural and non-natural compounds. The amino acid may be any of a neutral amino acid, a basic amino acid, and an acidic amino acid. In addition to an amino acid that itself functions as a transmitter such as a neurotransmitter, an amino acid that is a constituent of a physiologically active peptide (including a dipeptide, a tripeptide, a tetrapeptide, and an oligopeptide) or a polypeptide compound such as a protein can be used. For example, the amino acid may be an α-amino acid, a β-amino acid, a γ-amino acid, or the like. As the amino acid, an optically active amino acid is preferably used. For example, as the α-amino acid, either D- or L-amino acid may be used, however, it may be preferable to select an optically active amino acid that functions in a living body.

In the present specification, the “amino acid residue” refers to a structure corresponding to the remaining partial structure obtained by removing a hydroxyl group from a carboxyl group of an amino acid. The amino acid residue includes an α-amino acid residue, a β-amino acid residue, and a γ-amino acid residue. Examples of a preferred amino acid residue include a γ-glutamyl group of a GGT substrate and a dipeptide of a DPP4 substrate (a dipeptide composed of amino acid-proline).

In the present specification, the “peptide” refers to a structure in which two or more amino acids are linked by a peptide bond.

Examples of a preferred peptide include a dipeptide of the DPP4 substrate (dipeptide composed of amino acid-proline; where the amino acid is, for example, glycine, glutamic acid, or proline).

Examples of the case where L constitutes a part of an amino acid together with C═O to which L is bonded include a structure in which a carboxyl group of a side chain of an amino acid is bonded to —NH₂ to form a carbonyl group, which is a part of the amino acid, as in the γ-glutamyl group described above.

L′ is a saccharide or a partial structure of a saccharide, a saccharide having a self-cleaving linker, or an amino acid or a peptide having a self-cleaving linker.

The partial structure of the saccharide of L′ constitutes a saccharide or a part of a saccharide together with O to which L′ is bonded.

Examples of the saccharide include β-D-glucose, β-D-galactose, β-L-galactose, β-D-xylose, α-D-mannose, β-D-fucose, α-L-fucose, β-L-fucose, β-D-arabinose, β-L-arabinose, β-D-N-acetylglucosamine, and β-D-N-acetylgalactosamine, and β-D-galactose is preferable.

The self-cleaving linker means a linker that is spontaneously cleaved and decomposed, and examples thereof include a carbamate, a urea, a para-aminobenzyloxy group, and an ester group.

In one preferred aspect of the present invention, Y has a structure selected from the following.

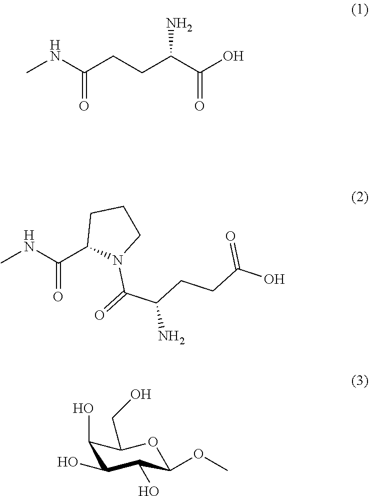

In General Formula (I), X acts as a leaving group that leaves from a benzene ring when a part of the enzyme recognition site of Y is cleaved by a cancer cell-specific enzyme activity, and as a result, a quinone methide is formed.

X is selected from the group consisting of a fluorine atom, an ester group (—OC(═O)—R′), a carbonate group (—OCO₂—R′), a carbamate group (—OCONH—R′), phosphoric acid and an ester group thereof (—OP(═O) (—OR′) (—OR″)), and sulfuric acid and an ester group thereof (—OSO₂—OR′).

Here, R′ and R′″ are each independently selected from a substituted or unsubstituted alkyl group or a substituted or unsubstituted aryl group.

X is preferably a fluorine atom or an ester group (—OC (═O)—R′). Although not intended to be bound by theory, in a case where X is a fluorine atom or an ester group (—OC (═O)—R′), a quinone methide is formed as soon as Y is cleaved.

R¹ and R² are each independently selected from a hydrogen atom or a monovalent substituent. The monovalent substituent is a halogen atom or an alkyl group having one or more carbon atoms (for example, an alkyl group having about 1 to 6 carbon atoms).

Preferably, R¹ and R² are each independently selected from a hydrogen atom or a fluorine atom.

—Y in General Formula (I) is preferably bonded to —C(R¹)(R²) X at an ortho position or a para position of a benzene ring. When —Y and —C(R¹) (R²) X are in such a positional relationship on the benzene ring, a quinone methide structure can be formed when Y is cleaved.

R³ is a hydrogen atom or one to three identical or different monovalent substituents present on a benzene ring.

The monovalent substituent of R³ is selected from the group consisting of an alkyl group having one or more carbon atoms (for example, an alkyl group having about 1 to 6 carbon atoms), an alkoxycarbonyl group (—C(═O)—OR′), a nitro group, an amino group, a hydroxyl group, an alkylamino group (—NHR′ or —NR′₂), an alkoxy group (—OR′), an ester group (−O—CO—R′), an amide group (—NHCOR′), a halogen atom, a boryl group, and a cyano group. Here, R′ is a substituted or unsubstituted alkyl group or a substituted or unsubstituted aryl group. When there are two or more R's, R's may be the same as or different from each other.

In one aspect of the compound of the present invention, the monovalent substituent of R³ is an alkyl group (for example, a methyl group) or an alkoxy group (for example, a methoxy group). When an alkyl group or an alkoxy group which is an electron-donating group is introduced into a benzene ring, it is preferable because the intracellular retention is excellent.

In one aspect of the compound of the present invention, the monovalent substituent of R³ is a halogen atom (preferably, an iodine atom). In a case where R³ is a halogen atom (preferably, an iodine atom), it is possible to enhance the trapping effect on cells.

In one aspect of the compound of the present invention, one or more of the monovalent substituents of R³ are alkyl groups (for example, methyl groups) or alkoxy groups (for example, methoxy groups), and one or more of the monovalent substituents of R³ are halogen atoms.

In a case where R³ is the monovalent substituent described above, in particular, an alkyl group or an alkoxy group, the position of R³ is preferably the 5-position corresponding to the para position and/or the 4-position corresponding to the meta position of -C (R¹) (R²)X.

In another aspect of the compound of the present invention, all of R³s are hydrogen atoms.

In General Formula (I), B represents a group containing ¹⁰B. B may be any group as long as it is a group containing ¹⁰B, and may be a group derived from a compound having one boron atom in the molecule, such as a boric acid residue (¹⁰B(OH)₂—), and a group derived from a boron cluster is preferable.

The boron cluster may be any polyhedral structure that can be used in a boron neutron capture therapy. Examples thereof include closo-dodecaborate ([B₁₂H₁₂]²⁻), ionic closo-carborane ([CB₁₁H₁₂]⁻), fat-soluble closo-carborane ([C₂B₁₀H_{12]}), nidocarborane ([C₂B₉H₁₁]⁻), a bisdicarbolide metal complex ([(C₂B₉H₁₁)₂M] (M is a metal)), GB10 ([B₁₀H₁₂]²⁻), 1,2-dicarbacloso-dodecarborane, 1,7-dicarba-closo-dodecarborane, 1,12-dicarba-closo-dodecarborane, dicarba-closo-decarborane ([C₂B₈H₁₀]), and sulfur-substituted undecahydrododecaborate, but are not limited thereto.

The boron atoms contained in the boron cluster may be all ¹⁰B, and only some may be ¹⁰B.

Note that, in the present specification, the expression “group derived from” such as “group derived from a boron cluster” is used, and this means, for example, a group derived by removing one hydrogen atom in the boron cluster.

In General Formula (I), Z represents a single bond or a linking group.

Here, in a case where Z is a “single bond”, it means that B is directly bonded to a benzene ring without a linking group interposed therebetween.

As the linking group, any linking group may be used as long as it has a function as a linker and is metabolically stable, and the linking group is preferably selected from the group consisting of an alkylene group (where one or more —CH₂— of the alkylene group may be replaced by —O—, —S—, —NH—, or —CO—), arylene (including heteroarylene), cycloalkylene (for example, cyclohexylene), an alkoxyl group, a polyethylene glycol chain, and a group constituted by optionally binding two or more groups selected from these groups.

The number of carbon atoms in the alkylene group is not particularly limited, and is preferably 5 to 20, and more preferably 5 to 15. Note that, in a case where —CH₂— in the alkylene group is replaced by —O—, —S—, —NH—, or —CO—, these groups are considered to have one carbon and are included in the “number of carbon atoms in the alkylene group” described above.

In addition, arylene contains a linker having a benzene ring such as a phenylene group, or a divalent linker derived from an aromatic or cyclic hydrocarbon containing a heterocyclic ring.

In one preferred aspect of the compound of the present invention, the linking group is an alkylene group (where one or more —CH₂— of the alkylene group may be replaced by —O—, —S—, —NH—, or —CO—).

The position at which B—Z— is introduced is not particularly limited, and it is preferable that B—Z— is bonded to Y at the meta position or the para position of the benzene ring because B—Z— is metabolically stable and there is a possibility that B—Z— may not be a substrate of a target enzyme when B—Z— is too close to the enzyme recognition site.

Non-limiting examples of the compound of the present invention are shown below, but the compound of the present invention is not limited thereto.

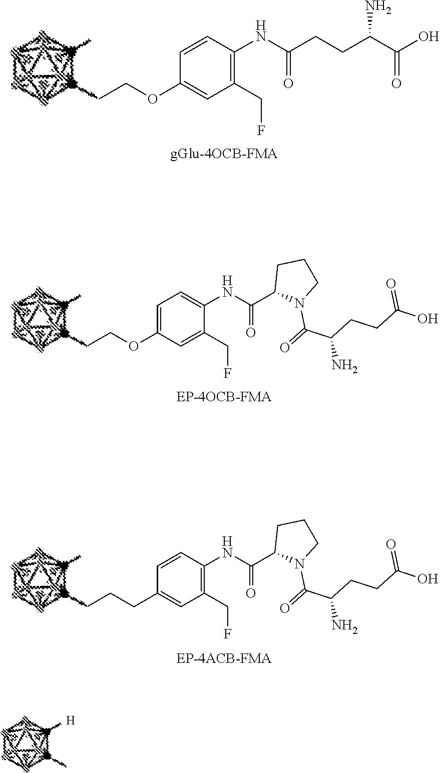

is a group derived from closo-carborane (o-carborane) (in the formula, a gray atom is BH, and a black atom is C).

Unless otherwise specified, the compound represented by General Formula (I) also contains stereoisomers such as tautomers, geometric isomers (for example, E-isomer, Z-isomer, and the like), and enantiomers thereof. That is, in a case where one or two or more asymmetric carbons are contained in the compound represented by General Formula (I), either the (R)-isomer or the (S)-isomer can be independently taken as stereochemistry of the asymmetric carbon, and the compound may exist as a stereoisomer such as an enantiomer or a diastereoisomer of the derivative. Therefore, as an active ingredient of the probe for the BNCT of the present invention, any stereoisomer in pure form, any mixture of stereoisomers, racemate, and the like can be used, and all of them are included in the scope of the present invention.

The method for producing a compound represented by General Formula (I) is not particularly limited, but a synthesis method for a representative compound among the compounds included in General Formula (I) is specifically shown in Examples of the present specification. Those skilled in the art can produce a compound included in Formula (I) by appropriately altering or modifying a starting material, a reaction reagent, reaction conditions, and the like as necessary while referring to Examples of the present specification and the following scheme.

### 2. Pharmaceutical Composition

Another embodiment of the present invention is a pharmaceutical composition comprising the compound of the present invention or a pharmaceutically acceptable salt thereof (hereinafter, also referred to as a “pharmaceutical composition of the present invention”).

A preferred aspect of the pharmaceutical composition of the present invention is a pharmaceutical composition used in a boron neutron capture therapy.

The pharmaceutical composition of the present invention is preferably a pharmaceutical composition used in a boron neutron capture therapy, which can be accumulated in cancer cells by acting selectively on cells by a cancer cell-specific enzyme activity.

A cancer cell-specific enzyme is a peptidase or a glycosidase.

Examples of the peptidase include Y-glutamyl transpeptidase (GGT), dipeptidyl peptidase IV (DPP-IV), and calpain.

Examples of the glycosidase include β-galactosidase, β-glucosidase, α-mannosidase, α-L-fucosidase, β-hexosaminidase, and β-N-acetylgalactosaminidase.

The pharmaceutical composition of the present invention may contain not only the compound represented by General Formula (I) but also a salt thereof or a solvate or a hydrate of the compound or the salt. The salt is not particularly limited as long as it is a pharmaceutically acceptable salt, and examples thereof include a base addition salt, an acid addition salt, and an amino acid salt. Examples of the base addition salt include alkaline earth metal salts such as a sodium salt, a potassium salt, a calcium salt, and a magnesium salt; an ammonium salt; and organic amine salts such as a triethylamine salt, a piperidine salt, and a morpholine salt, and examples of the acid addition salt include mineral salts such as a hydrochloride, a hydrobromide, sulfate, nitrate, and phosphate; and organic acid salts such as methanesulfonic acid, benzenesulfonic acid, p-toluenesulfonic acid, acetic acid, propionate, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, and salicylic acid. Examples of the amino acid salt include a glycine salt, an aspartic acid salt, and a glutamic acid salt. In addition, the salt may be a metal salt such as an aluminum salt.

The type of solvent for forming the solvate is not particularly limited, and examples thereof include solvents such as ethanol, acetone, and isopropanol.

The pharmaceutical composition of the present invention is used in a boron neutron capture therapy. That is, the pharmaceutical composition of the present invention is administered to a human or a non-human animal (a mouse, a rat, a hamster, a rabbit, a cat, a dog, a cow, a sheep, a monkey, or the like), and then performing irradiation with a low-energy thermal neutron to selectively destroying tumor cells. Examples of a disease to be treated include malignant tumors such as brain tumor, malignant melanoma, head and neck cancer, lung cancer, liver cancer, thyroid cancer, skin cancer, bladder cancer, mesothelioma, pancreatic cancer, breast cancer, meningioma, and sarcoma, but are not limited thereto.

In a case where the pharmaceutical composition is used as a pharmaceutical composition containing the compound represented by General Formula (I) or a pharmaceutically acceptable salt thereof, it can be formulated by mixing with a pharmaceutically acceptable carrier or diluent according to a known method. A dosage form is not particularly limited, and can be an injection, a tablet, a powder, a granule, a capsule, a liquid, a suppository, a sustained-release agent, or the like. The administration method is also not particularly limited, and the pharmaceutical compound can be administered orally or parenterally (administration by intradermal, intraperitoneal, intravenous, arterial, or spinal fluid injection, infusion, or the like).

These formulations are prepared according to a conventional method. Note that the liquid preparation may be dissolved or suspended in water or another appropriate solvent at the time of use. In addition, the tablet and the granule may be coated by a well-known method. In the case of the injection, the injection is prepared by dissolving the compound of the present invention in water, and may be dissolved in physiological saline or a glucose solution as necessary, or a buffer or a preservative may be added.

A dosage of the pharmaceutical compound of the present invention varies depending on a subject to be administered, an administration method, and the like. For example, in a case where the pharmaceutical composition is administered as an injection to an adult, the pharmaceutical composition can be administered in one to several divided doses in one treatment so that the compound is 10 to 1,000 mg/kg per one dose.

Another embodiment of the present invention is a method for diagnosing, treating, or diagnosing and treating a disease or a symptom that may cause the disease, the method (hereinafter, also referred to as a “method of the present invention”) including:

(A) administering the pharmaceutical composition of the present invention to a subject having or suspected of having a disease or a symptom; and
(B) irradiating a ¹⁰B atom localized in a target tissue of the subject with a neutron beam to perform a boron neutron capture therapy on the target tissue.

In the method of the present invention, a dosage of the pharmaceutical composition of the present invention is as described above.

In the method of the present invention, in order to irradiate the ¹⁰B atom localized in a target tissue of a subject with a neutron beam, various conditions required for a treatment, such as a neutron dose, a neutron spectrum, and an irradiation time, are determined using a nuclear reactor or an accelerator-type neutron generator usually used in the BNCT. The energy of the neutron beam to be radiated is usually about 0.025 eV for a thermal neutron and 0.5 eV to 40 keV for an epithermal neutron.

### EXAMPLES

Hereinafter, the present invention will be described using Examples, but the present invention is not limited thereto.

### General Procedure and Materials

All reagents and dry solvents were purchased from commercial suppliers (Tokyo Chemical Industry Co., Ltd., FUJIFILM Wako Pure Chemical Corporation, Sigma-Aldrich, Kanto Chemical Co., Inc., Dojindo Laboratories Co., Ltd., Watanabe Chemical Co., Ltd., Gibco, Invitrogen, Thermo scientific, and Merck Inc.) and were used without further purification.

### Used Instrument

Reaction progress was observed with TLC silica gel 60F254 (Merck Inc.) and ACQUITY UPLC/MS system (Waters Inc.).

¹HNMR and ¹³CNMR spectra were measured with JEOL JNM-ECZ 400 (400 MHz for ¹HNMR and 100 MHz for ¹³CNMR); and a σ value was ppm with respect to tetramethylsilane (TMS).

Mass spectra (MS) were measured with JEOL JMS-T100 LC AccuToF (ESI).

Column chromatography using silica gel was performed with MPLC system (Yamazen Smart Flash EPCLC W-Prep 2XY (Tokyo, Japan)).

Reverse phase MPLC purification was performed with Isolera™ One (Biotage) equipped with SNAP Ultra C18 (Biotage).

Preparative HPLC was performed with Inertsil ODS-3 (10.0×250 mm) column (GL Sciences Inc.) using an HPLC system including a pump (PU-2086 (JASCO)) and a detector (MD-2015 or FP-2025, JASCO) using an eluent A (H₂O with 0.1% TFA (v/v)) and an eluent B (CH₃CN with 20% H₂O with 0.1% TFA (v/v)), or an eluent C (H₂O with 100 mM TEAA, that is, 100 mM trimethylamine and acetic acid aqueous solution) and an eluent D (CH₃CN with 20% H₂O with 100 mM TEAA) at a flow rate of 3 or 5 mL/min.

### Enzyme Assay (LC/MS Assay)

In a screw-capped vial for LC/MS assay (Agilent Technologies Inc.), phosphate buffered saline (pH 7.4) or a 0.1 M HEPES buffer (pH 7.4) containing a prodrug (100 μM) in the presence or absence of an inhibitor (100 μM with respect to GGT and 200 μM of GGsTop (registered trademark) with respect to DPP-IV) and DMSO (1%) as a co-solvent was prepared. An enzyme (1 U/mL for GGT and >0.1 mU/mL for DPP-IV) was added, and then culture was performed at 37° C. for 12 hours. LC/MS assay (SIM) was performed under the following conditions (eluent A: H₂O with 0.01 M ammonium formate, eluent B: H₂O with 80% acetonitrile/0.01 M ammonium formate, A/B=90/10→0/100 in 12 minutes). In an experiment for evaluating the reaction of an azaquinone methide with a nucleophilic reagent (GSH, 1-Cys), a 0.1 M HEPES buffer (pH 7.4) containing 5 mM of each nucleophile was prepared and used.

### Cell Culture

H226, H460, SHIN3, and SKOV3 cells were cultured in RPMI 1640 medium (Roswell Park Memorial Institute 1640 medium, Gibco) containing 10% fetal bovine serum (Gibco) and 1% penicillin streptomycin (Gibco). A549, Hela, and HepG2 cells were cultured in Dulbecco's Modified Eagle Medium (DMEM, Gibco) containing 10% fetal bovine serum and 1% penicillin streptomycin. Caco-2 cells were cultured in DMEM containing 20% fetal bovine serum, 1% penicillin streptomycin, and 1% MEM non-essential amino acid solution (100×). All the cells were cultured in an incubator with 5% CO₂ at 37° C.

### Cell Viability Test (CCK-8 Assay)

Cells (1.0×104 cells/well) were proliferated in a 96-well dish for 24 hours and treated with a prodrug over a range of concentrations (0 [control] or 1, 2.5, 5, 10, 25, and 50). After 24 hours, a degree of cell proliferation was evaluated using CCK-8 assay (Tokyo, Japan, Dojindo Laboratories Co., Ltd.). CCK-8 solution (10 μL) was added to each well, and subsequently, incubation was performed in 5% CO₂ at 37° C. for 2 hours. An absorbance at 440 nm was measured by Envision 2103 multilabel reader (Prekin Elmer). Cell viability was expressed as a percentage of control cells. For each concentration of the prodrug, an average value of average absorption rates from the three wells was calculated.

### Uptake of Cells

In order to evaluate cellular uptake, H226 cells were seeded in a 6-well dish (2.5×10⁵ cells/mL, 5.0×10⁵ cells/well) and incubated for 24 hours. After removing the medium, the cells were incubated in a medium containing EP-4OCB-FMA (10 μM) and 1% DMSO in the presence or absence of sitagliptin (100 μM) for 3 hours. Next, 100 μL of a supernatant was collected and diluted with 900 μL of 5.5% nitric acid to be used as a sample “supernatant”. After removing the remaining supernatant, the cells were washed with PBS and detached by incubating in 0.2 ml of a 0.05% trypsin/EDTA solution. A cell suspension was mixed with 2 mL of a medium and cells were recovered by centrifugation. After carefully removing the supernatant, 2 mL of a medium was added, and the number of cells was measured. Next, the cells were dissolved in 400 μL of 60% nitric acid and the dissolved matter was heated to 90° C. to be ashed. The ashed sample was diluted with 4.4 mL of ultrapure water and used as a sample “cell”. The amount of boron in the sample was quantified using MP-AES (Agilent 4100 MP-AES, Agilent Technologies Inc., Santa Clara, California). In an experiment for evaluating intracellular boron retention, cells were first incubated with EP-4OCB-FMA without sitagliptin for 3 hours as described above. Next, the cells were incubated in a fresh medium without EP-4OCB-FMA for 30 minutes to release intracellular boron, and subsequently, the cells were recovered with 0.05% trypsin/EDTA. The subsequent procedure was the same as the measurement method described above.

### Synthesis Example 1

According to the following Synthesis Scheme 1, gGlu-4OCB-FMA (compound 18) as the compound of the present invention was synthesized as a GGT candidate drug. Details of each reaction step will be described below.

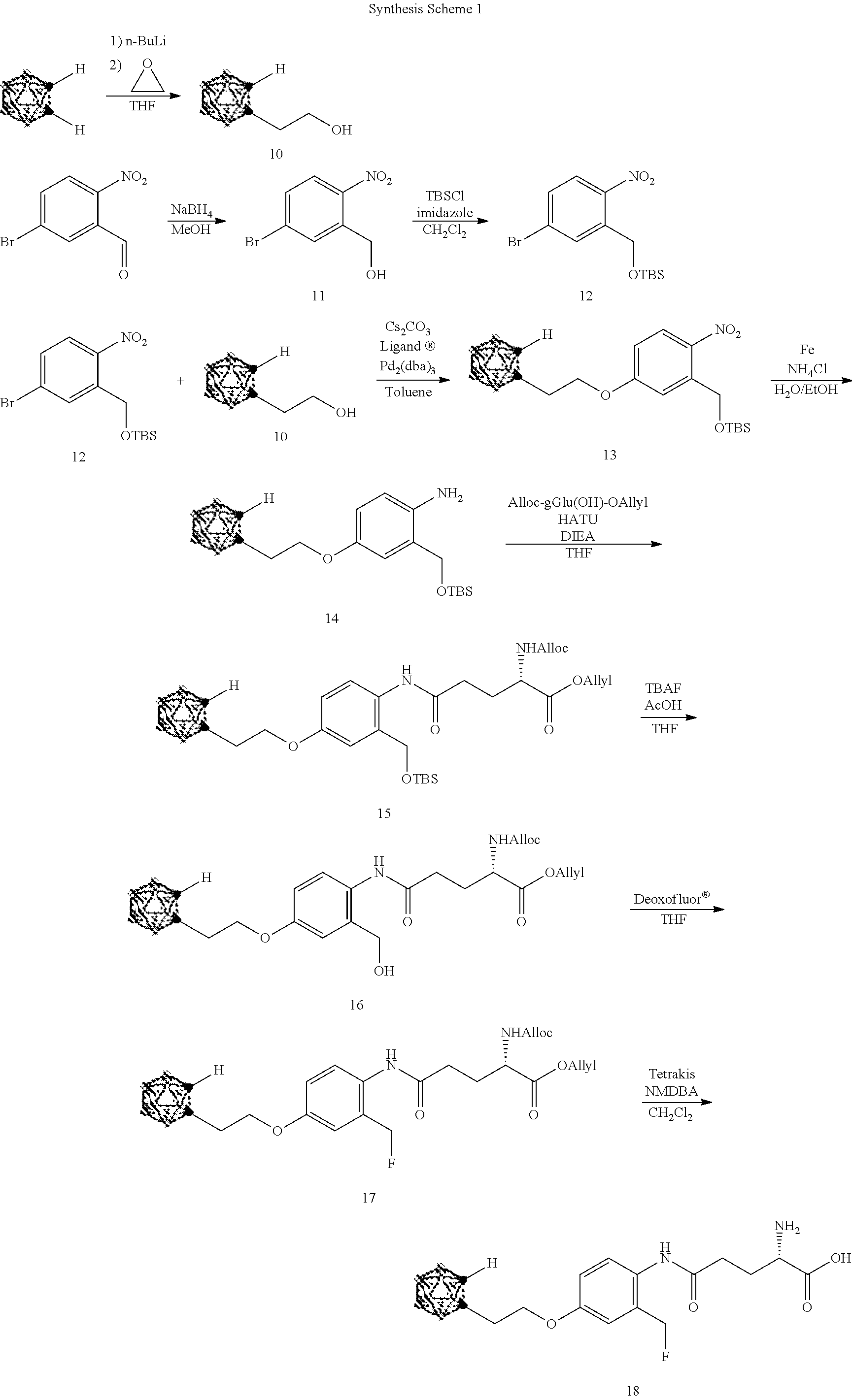

### (1) Synthesis of Compound 10

o-Carborane (534 mg, 3.70 mmol) was dissolved in 12 mL of dehydrated THE. After cooling to −78° C., a THE solution of 1.6 Mn-BuLi (2.5 mL, 4.0 mmol) was added dropwise to the solution. After stirring in an argon atmosphere at −78° C. for 30 minutes, a THE solution (5.0 mL, 6.0 mmol) of 1.2 M ethylene oxide was added dropwise for 10 minutes, and then the reaction solution was heated to 0° C. After stirring for 1 hour, 4 mL of a NH₄Cl saturated aqueous solution was added. After stirring for several 10 minutes, the reaction solution was extracted three times with AcOEt. The combined organic layers were dried with Na₂SO₄ and concentrated under low pressure. The residue was purified by MPLC (OH silica gel, AcOEt/hexane) to obtain light yellow oily matter (375.3 mg, 1.98 mmol, 54%). ¹H-NMR (400 MHz, CDCl₃) δ 3.98 (s, 1H), 3.79 (t, J=5.9 Hz, 2H), 2.49 (t, J=5.9 Hz, 2H), 1.68 (s, 1H), 1.2-3.2 (br, 10H) ¹³C-NMR (101 MHz, CDCl₃) 873.10, 60.74, 60.49, 39.85

### (2) Synthesis of Compound 11

5-Bromo-2-nitrobenzaldehyde (1.00 g, 4.35 mmol) was dissolved in 20 mL of dry MeOH, and then sodium borohydride (163 mg, 4.30 mmol) was partially added at 0° C. The solution was stirred at room temperature for 30 minutes. The solution was heated to room temperature, and then stirring was continued for 10 minutes. The reaction was quenched with water, and then the solvent was evaporated and concentrated. The mixture was extracted with AcOEt, and then the organic layer was washed with a saline solution, dried with Na₂SO₄, and concentrated under low pressure. The residue was purified by MPLC (OH silica gel, AcOEt/hexane) to obtain a white solid (1,211 mg, 5.22 mmol, quantitative yield).

### (3) Synthesis of Compound 12

The compound 11 (602 mg, 2.59 mmol), TBSCl (781 mg, 5.18 mmol), and imidazole (531 mg, 7.80 mmol) were added to 25 mL of dry toluene and 5 mL of dry CH₂Cl₂. The solution was stirred in an argon atmosphere at room temperature for 16 hours. AcOEt was added to the solvent, and the organic layer was washed twice with water. The organic layer was dehydrated with Na₂SO₄ and concentrated under low pressure. The residue was purified by MPLC (OH silica gel, AcOEt/hexane) to obtain an orange oil (838 mg, 2.42 mmol, 93%).

### (4) Synthesis of Compound 13

The compound 12 (401 mg, 1.16 mmol) and the compound 10 (283 mg, 1.50 mmol) were dissolved in 2 mL of dry toluene, and then Cs₂CO₃ (549 mg, 1.75 mmol) and 2-di-t-butylphosphino-2′,4′,6′-triisopropyl-1,1′-biphenyl (6.4 mg, 0.015 mmol) were added. After degassing by a freeze-pump-thaw cycle, Pd₂ (dba)₃ (6.9 mg, 0.0075 mmol) was added to the solution. The reaction solution was stirred in an argon atmosphere at 85° C. for 2 hours. AcOEt was added to the solvent, and the organic layer was washed with water. The organic layer was dehydrated with Na₂SO₄ and concentrated under low pressure. The residue was purified by MPLC (OH silica gel, AcOEt/hexane) to obtain an orange solid (384 mg, 0.845 mmol, 73%).

¹H-NMR (400 MHz, CDCl₃) δ 8.18 (d, J=9.0 Hz, 1H), 7.42 (d, J=3.2 Hz, 1H), 6.82 (dd, J=9.0, 3.2 Hz, 1H), 5.11 (s, 2H), 4.17 (t, J=6.2 Hz, 2H), 3.79 (s, 1H), 2.79 (t, J=6.2 Hz, 2H), 1.40-3.20 (br, 10H), 1.00 (s, 9H), 0.16 (s, 6H). ¹³C-NMR (101 MHz, CDCl₃) δ 162.1, 142.5, 140.1, 127.8, 113.1, 112.7, 72.1, 66.1, 62.4, 60.8, 37.1, 26.1, 18.5, −5.3; HRMS Calcd for ¹²C₁₇¹H₃₄¹⁰B₂¹¹B₈¹⁴N¹⁶O₄²⁸Si:452.32603 [M-H]⁻; Found: 452.32608 (+0.05 mDa).

### (5) Synthesis of Compound 14

The compound 13 (384 mg, 0.846 mmol) was dissolved in 13 mL of ethanol and 12 mL of water, and then ammonium chloride (144 mg, 2.68 mmol) and Fe (195 mg, 3.49 mmol) were added. After stirring at room temperature for 1 hour, the solvent was stirred at 75° C. for 3 hours. Ethanol was removed by evaporation. The remaining solution was extracted with AcOEt and the organic layer was washed with a NaHCO₃ saturated aqueous solution. The organic layer was dehydrated with Na₂SO₄ and concentrated under low pressure. The residue was purified by MPLC (OH silica gel, AcOEt/hexane) to obtain an orange solid (196 mg, 0.463 mmol, 55%).

¹H-NMR (400 MHz, CDCl₃) δ 6.61-6.64 (m, 3H), 4.63 (s, 2H), 3.98 (t, J=5.7 Hz, 2H), 3.90 (s, 1H), 2.68 (t, J=5.7 Hz, 2H), 1.40-3.20 (br, 10H), 0.91 (s, 9H), 0.09 (s, 6H). ¹³C-NMR (101 MHz, CDCl₃) δ 150.2, 140.4, 127.0, 116.9, 115.2, 114.6, 73.0, 66.4, 64.4, 60.3, 37.41, 26.0, 18.4, −5.4

### (6) Synthesis of Compound 15

The compound 14 (177 mg, 0.418 mmol) and N-Alloc-Glu(OH)-OAllyl (141 mg, 0.520 mmol) were dissolved in 6 mL of dry THF, and subsequently, HATU (490 mg, 1.29 mmol) and DIEA (225 μL, 1.29 mmol) were added. The solution was stirred in an argon atmosphere at room temperature for 8 hours. The solvent was removed by evaporation. AcOEt was added to the residue, and the organic layer was washed with water. The organic layer was dried with Na₂SO₄ and concentrated under low pressure. The residue was purified by MPLC (OH silica gel, AcOEt/hexane) to obtain a white solid (276 mg, 0.408 mmol, 98%).

¹H-NMR (400 MHz, CDCl₃) δ 8.64 (s, 1H), 7.99 (d, J=8.8 Hz, 1H), 6.76 (dd, J=8.8, 3.0 Hz, 1H), 6.66 (d, J=3.0 Hz, 1H), 5.84-5.94 (m, 2H), 5.63 (d, J=7.9 Hz, 1H), 5.18-5.35 (m, 4H), 4.68 (s, 2H), 4.65 (d, J=5.0 Hz, 2H), 4.56 (d, J=4.6 Hz, 2H), 4.43 (m, 1H), 4.04 (t, J=5.7 Hz, 2H), 3.84 (s, 1H), 2.71 (t, J=5.7 Hz, 2H), 2.31-2.53 (m, 3H), 2.07-2.15 (m, 1H), 0.92 (s, 9H), 0.10 (s, 6H)

### (7) Synthesis of Compound 16

The compound 15 (243 mg, 0.359 mmol) was dissolved in 20 mL of dry THF, and subsequently, TBAF (in a THF solution, about 1 M) (914 μL, 0.914 mmol) and acetic acid (34.9 μL, 0.610 mmol) were added. The solution was stirred in an argon atmosphere at room temperature for 45 minutes, and the solvent was removed by evaporation. AcOEt was added to the residue, and the organic layer was washed with water. The organic layer was dehydrated with Na₂SO₄ and concentrated under low pressure. The residue was purified by MPLC (OH silica gel, AcOEt/hexane) to obtain a white solid (132 mg, 0.235 mmol, 65%).

¹H-NMR (400 MHz, CDCl₃) δ 8.49 (s, 1H), 7.73 (d, J=8.2 Hz, 1H), 6.77-6.80 (m, 2H), 5.83-5.95 (m, 2H), 5.70 (brs, 1H), 5.20-5.36 (m, 4H), 4.54-4.65 (m, 6H), 4.42 (m, 1H), 4.04 (t, J=5.7 Hz, 2H), 3.85 (s, 1H), 3.22 (brs, 1H), 2.71 (t, J=5.7 Hz, 2H), 2.33-2.50 (m, 3H), 1.98-2.07 (m, 1H), 1.40-3.20 (br, 10H). ¹³C-NMR (101 MHz, CDCl₃) δ 171.68, 171.03, 156.57, 154.78, 132.40, 131.37, 130.47, 125.33, 119.36, 118.20, 115.28, 114.10, 72.68, 66.42, 66.21, 65.79, 63.59, 60.41, 53.58, 37.21, 33.46, 29.00.

### (8) Synthesis of Compound 17

The compound 16 (48 mg, 0.085 mmol) was dissolved in 3 mL of dry CH₂Cl₂, and then 2 mL of a dry CH₂Cl₂ solution of Deoxofluor (registered trademark) (31.6 μL, 0.171 mmol) was added at 0° C. The solution was stirred in an argon atmosphere at room temperature for 30 minutes, and CH₂C₁₂ was added to the reaction solution and washed with a NaHCO₃ saturated aqueous solution. The organic layer was dehydrated with Na₂SO₄ and concentrated under low pressure. The residue was purified by MPLC (OH silica gel, AcOEt/hexane) to obtain a white solid (34 mg, 0.060 mmol, 71%).

¹H-NMR (400 MHz, CDCl₃) δ 7.80 (s, 1H), 7.63 (d, J=9.1 Hz, 1H), 6.85 (m, 2H), 5.85-5.95 (m, 2H), 5.63 (d, J=7.3 Hz, 1H), 5.20-5.47 (m, 6H), 4.65 (d, J=5.9 Hz, 2H), 4.57 (d, J=5.5 Hz, 2H), 4.44-4.47 (m, 1H), 4.06 (t, J=5.7 Hz, 2H), 3.83 (s, 1H), 2.72 (t, J=5.7 Hz, 2H), 2.50 (m, 2H), 2.31-2.38 (m, 1H), 1.96-2.07 (m, 1H), 1.40-3.20 (br, 10H). ¹³C-NMR (101 MHz, CDCl₃) δ 171.64, 170.83, 156.42, 155.17, 132.49, 131.40, 130.57, 130.43, 129.64, 126.46, 119.31, 118.13, 115.28, 114.84, 114.77, 83.44, 81.80, 72.59, 66.37, 66.15, 65.88, 60.42, 53.50, 37.22, 33.11, 29.00.

### (9) Synthesis of Compound 18

The compound 17 (17 mg, 0.030 mmol) was dissolved in 5 mL of dry CH₂Cl₂, and then 1, 3-dimethylbarbituric acid (46 mg, 0.30 mmol) and tetrakis (9.7 mg, 0.084 mmol) were added. The solution was stirred in an argon atmosphere at room temperature for 4 hours. The reaction solution was concentrated by evaporation. The residue was purified by HPLC (eluent: A: H₂O, 0.1% TFA (v/v), eluent: B: CH₃CN/H₂O=80/20, 0.1% TFA (v/v)) to obtain a white solid (8.6 mg, 0.020 mmol, 65%).

¹H-NMR (400 MHz, MeOH-D4) δ 7.25 (d, J=8.7 Hz, 1H), 7.03 (d, J=2.7 Hz, 1H), 6.94 (dd, J=8.7, 2.7 Hz, 1H), 5.34 (d, J=47.6 Hz, 2H), 4.59 (s, 1H), 4.11 (t, J=5.8 Hz, 2H), 4.03 (t, J=6.4 Hz, 1H), 2.78 (t, J=5.8 Hz, 2H), 2.70 (t, J=7.3 Hz, 2H), 2.19-2.30 (m, 2H). ¹³C-NMR (101 MHz, MeOH-D4) δ 172.32, 170.35, 156.73, 133.89, 133.72, 127.83, 114.65, 114.63, 113.74, 113.66, 81.75, 80.12, 73.36, 65.67, 61.94, 52.26, 36.77, 31.05, 25.78.

### Example 1

Confirmation of Reactivity of gGlu-4OCB-FMA with Purified Enzyme

In order to verify whether gGlu-4OCB-FMA synthesized above can be a substrate for GGT, an enzymatic reaction with a purified enzyme was performed, and product assay was performed by LC/MS.

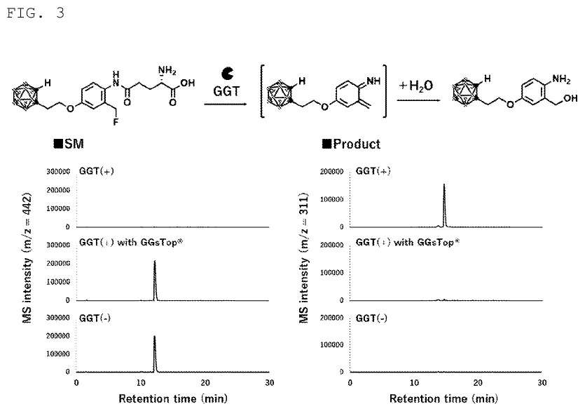

As a result, gGlu-4OCB-FMA was completely consumed by addition of GGT, and a benzyl alcohol form was confirmed as a product (FIG. 3). This is considered to be due to the reaction of the generated azaquinone methide intermediate with the water molecules in the buffer as nucleophiles, which indirectly indicates the generation of an azaquinone methide intermediate. In addition, since the enzymatic reaction did not proceed in the presence of GGsTop (registered trademark) as an inhibitor of GGT, it was confirmed that gGlu-4OCB-FMA was a substrate of GGT. FIG. 3 illustrates LC/MS assay of the enzymatic reaction of gGlu-4OCB-FMA (100 UM): the results of mass chromatogram of gGlu-4OCB-FMA and the product compound after incubation at 37° C. for 12 hours in the presence or absence of GGT (1 U/mL), in the presence or absence of GGsTop (registered trademark) (100 μM), and in PBS (pH 7.4) (1% DMSO as a co-solvent).

### Synthesis Example 2

Next, the target enzyme of the candidate compound was changed to dipeptidyl peptidase IV (DPP-IV) as an aminopeptidase reported to have enhanced activity in various cancer cells, and based on the same drug design, EP-4OCB-FMA (compound 25) as the compound of the present invention was synthesized according to the following Synthesis Scheme 2. Here, DPP-IV is known to recognize various amino acid 2 residues as a substrate, but the Glu-Pro sequence is considered to be a highly water-soluble structure among them. Thus, the present sequence was adopted as a substrate site.

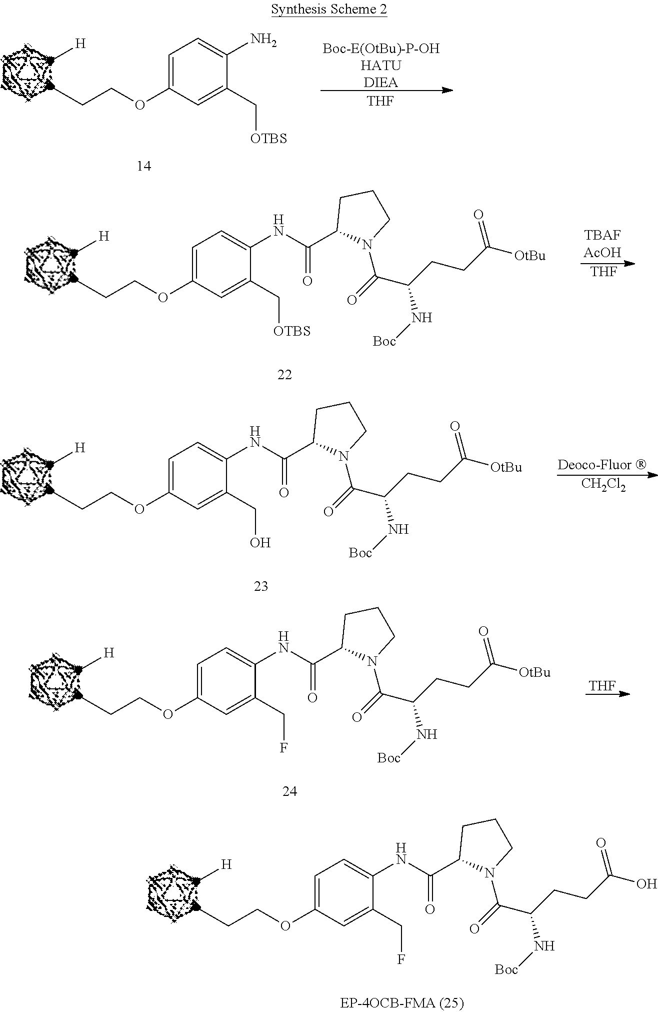

### (1) Synthesis of Compound 22

The compound 14 (125.4 mg, 0.2960 mmol) and Boc-E(OtBu)—P—OH (142.4 mg, 0.3556 mmol) were dissolved in 2 mL of dry THF, and subsequently, HATU (341.2 mg, 0.8973 mmol) and DIEA (155 μL, 0.888 mmol) were added. The solution was stirred in an argon atmosphere at room temperature for 3 hours. The solvent was removed by evaporation. AcOEt was added to the residue, and the organic layer was washed with a saline solution. The organic layer was dried with Na₂SO₄ and concentrated under low pressure. The residue was purified by MPLC (OH silica gel, AcOEt/hexane) to obtain a white solid (227.8 mg, 0.283 mmol, 96%).

¹H-NMR (400 MHz, CDCl₃) δ 8.82 (s, 1H), 7.81 (d, J=8.7 Hz, 1H), 6.80 (d, J=2.7 Hz, 1H), 6.72 (dd, J=8.7, 2.7 Hz, 1H), 5.23 (d, J=9.1 Hz, 1H), 4.75 (d, HBn, J_{gem}=13.3 Hz, 1H), 4.51-4.64 (m, 3H), 4.03 (t, J=5.7 Hz, 2H), 3.85 (s, 1H), 3.75-3.78 (m, 2H), 2.71 (t, J=5.7 Hz, 2H), 2.27-2.42 (m, 3H), 2.15-2.24 (m, 1H), 1.98-2.11 (m, 3H), 1.69-1.79 (m, 1H), 1.44 (s, 9H), 1.43 (s, 9H), 1.40-3.20 (br, 10H), 0.93 (s, 9H), 0.13 (s, 3H), 0.09 (s, 3H). ¹³C-NMR (101 MHz, CDCl₃) δ 172.20, 172.09, 169.37, 155.67, 154.33, 133.45, 130.01, 124.25, 113.72, 113.14, 80.69, 79.82, 72.74, 65.75, 63.44, 60.88, 60.28, 51.22, 47.48, 37.19, 30.98, 28.41, 28.30, 28.16, 28.03, 25.94, 25.25, 18.36, −5.12; LRMS 806.58 [C₃₆H₆₇¹⁰B₂¹¹B₈N₃O₈Si+H]⁺

### (2) Synthesis of Compound 23

The compound 22 (203 mg, 0.252 mmol) was dissolved in 15 mL of dry THF, and subsequently, TBAF (in a THF solution, about 1 M) (757 μL, 0.757 mmol) and AcOH (28.9 μL, 0.505 mmol) were added. The solution was stirred in an argon atmosphere at room temperature for 45 minutes, and the solvent was removed by evaporation. AcOEt was added to the residue, and the organic layer was washed with a saline solution. The organic layer was dehydrated with Na₂SO₄ and concentrated under low pressure. The residue was purified by MPLC (OH silica gel, AcOEt/hexane) to obtain a white solid (109.6 mg, 0.158 mmol, 63%).

¹H-NMR (400 MHz, CDCl₃) δ 9.10 (s, 1H), 7.78 (d, J=8.2 Hz, 1H), 6.74-6.78 (m, 2H), 5.29 (d, J=8.2 Hz, 1H), 4.73 (dd, J=7.8, 2.7 Hz, 1H), 4.55-4.58 (m, 3H), 4.03 (t, J=5.5 Hz, 2H), 3.73-3.84 (m, 4H), 2.71 (t, J=5.5 Hz, 2H), 2.05-2.41 (m, 5H), 1.78-1.85 (m, 1H), 1.44 (s, 9H), 1.41 (s, 9H), 1.40-3.20 (br, 10H). ¹³C-NMR (101 MHz, CDCl₃) δ 173.16, 172.66, 169.73, 155.55, 154.56, 133.28, 130.50, 124.81, 115.15, 113.91, 81.37, 80.08, 72.72, 65.75, 63.55, 61.24, 60.28, 51.22, 47.65, 37.22, 30.91, 28.41, 28.30, 28.08, 27.89, 25.17

### (3) Synthesis of Compound 24

The compound 23 (96.5 mg, 0.139 mmol) was dissolved in 6 mL of dry CH₂Cl₂, and then Deoxofluor (registered trademark) (51.4 μL, 0.279 mmol) was added at 0° C. The solution was stirred in an argon atmosphere at room temperature for 1 hour. CH₂Cl₂ was added to the reaction solution and washed with a NaHCO₃ saturated aqueous solution. The organic layer was dehydrated with Na₂SO₄ and concentrated under low pressure. The residue was purified by MPLC (OH silica gel, AcOEt/hexane) to obtain a transparent solid (79.8 mg, 0.115 mmol, 83%).

¹H-NMR (400 MHz, CDCl₃) δ 8.81 (s, 1H), 7.67 (d, J=8.2 Hz, 1H), 6.84 (m, 2H), 5.27-5.39 (m, 3H), 4.76 (dd, J=7.8, 2.3 Hz, 1H), 4.55 (td, J=9.0, 3.8 Hz, 1H), 4.05 (t, J=5.7 Hz, 2H), 3.84 (s, 1H), 3.74 (m, 2H), 1.40-3.20 (br, 10H), 2.72 (t, J=5.7 Hz, 2H), 2.46-2.51 (m, 1H), 2.27-2.43 (m, 2H), 1.95-2.20 (m, 4H), 1.75-1.81 (m, 1H), 1.44 (s, 9H), 1.42 (s, 9H). ¹³C-NMR (101 MHz, CDCl₃) δ 173.22, 172.20, 169.53, 155.63, 154.93, 130.00, 129.62, 125.78, 115.22, 114.80, 114.72, 82.85, 81.20, 80.86, 79.98, 72.63, 65.83, 60.70, 60.34, 51.21, 47.67, 37.20, 31.03, 28.40, 28.13, 28.01, 27.24, 25.30; LRMS 806.58 [C₃₀H₅₂¹⁰B₂¹¹B₈ FN₃O₇+H]⁺

### (4) Synthesis of Compound 25

The compound 24 (50.8 mg, 0.0732 mmol) was dissolved in 1 mL of TFA. The solution was stirred at room temperature for 10 minutes. The reaction solution was diluted with CH₂Cl₂ and concentrated by evaporation. The residue was purified by HPLC (eluent: A: H₂O, 0.1% TFA (v/v), eluent: B: CH₃CN/H₂O=80/20, 0.1% TFA (v/v)) to obtain a white solid (34.3 mg, 0.0638 mmol, 87%).

¹H-NMR (400 MHz, MeOH-D4) δ 7.22 (d, J=8.7 Hz, 1H), 7.03 (d, J=2.7 Hz, 1H), 6.93 (dd, J=8.7, 2.3 Hz, 1H), 5.24-5.48 (m, 2H), 4.58-4.63 (m, 2H), 4.38 (dd, J=7.3, 5.0 Hz, 1H), 4.10 (t, J=5.7 Hz, 2H), 3.69-3.78 (m, 2H), 1.40-3.20 (br, 10H), 2.77 (t, J=5.7 Hz, 2H), 2.59 (t, J=7.3 Hz, 2H), 2.36-2.42 (m, 1H), 2.00-2.24 (m, 5H). ¹³C-NMR (101 MHz, Acetonitrile-d3) δ 175.16, 170.76, 167.49, 156.20, 133.20, 133.04, 128.19, 128.14, 127.32, 115.00, 114.23, 114.15, 82.27, 80.65, 73.83, 65.80, 62.04, 60.93, 51.51, 47.49, 36.66, 29.11, 28.98, 24.98, 24.84; HRMS Calcd for ¹²C₂₁¹H₃₇¹⁰B₂¹¹B₈¹⁹F¹⁴N₃¹⁶O₅: 538.37204 [M+H]⁺; Found: 538.37055 (−1.49 mDa).

### Reference Synthesis Example 1

EP-4OCB-MA (compound 26) as a control compound was synthesized by the following Synthesis Scheme 3. Details of each reaction step will be described below.

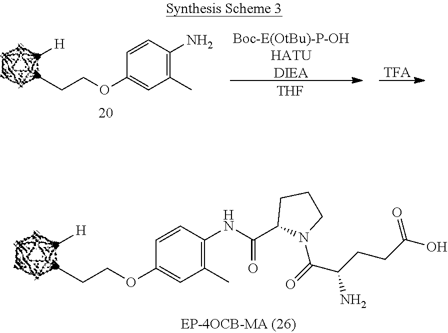

### (1) Synthesis of Compound 26

The compound 20 (62 mg, 0.21 mmol) and N-Boc-E(OtBu)—P—OH (124 mg, 0.310 mmol) were dissolved in several mL of dry THF, and subsequently, HATU (241 mg, 0.634 mmol) and DIEA (110.5 μL, 0.633 mmol) were added. The solution was stirred in an argon atmosphere at room temperature for 3 hours. The solvent was removed by evaporation. AcOEt and water were added to the residue, and the organic layer was extracted. The organic layer was dried with Na₂SO₄ and concentrated under low pressure. The crude compound was dissolved in 2 mL of TFA. The solution was stirred at room temperature for 10 minutes, and the reaction solution was concentrated by evaporation. The residue was purified by HPLC (eluent: A: H₂O, 0.1% TFA (v/v), eluent: B: CH₃CN/H₂=80/20, 0.1% TFA (v/v)) to obtain a white solid (84 mg, 0.16 mmol, 76%, 2 steps).

¹H-NMR (400 MHz, MeOH-D4) δ 7.16 (d, J=8.6 Hz, 1H), 6.80 (d, J=2.7 Hz, 1H), 6.73 (dd, J=8.6, 2.7 Hz, 1H), 4.65 (dd, J=8.2, 5.5 Hz, 1H), 4.58 (s, 1H), 4.38 (dd, J=7.3, 5.0 Hz, 1H), 4.05 (t, J=5.9 Hz, 2H), 3.68-3.79 (m, 2H), 2.75 (t, J=5.7 Hz, 2H), 2.59 (t, J=7.1 Hz, 2H), 2.36-2.41 (m, 1H), 2.02-2.26 (m, 8H), 1.40-3.20 (br, 10H). ¹³C-NMR (101 MHz, MeOH-D4) δ 174.66, 171.77, 167.22, 156.59, 135.64, 128.52, 127.35, 115.93, 111.69, 73.46, 65.44, 61.86, 60.58, 50.92, 48.31, 48.10, 47.88, 47.67, 47.46, 47.25, 47.03, 36.80, 29.60, 28.12, 25.28, 24.91, 17.05

### Example 2

Confirmation of Reactivity of EP-4OCB-FMA with Purified Enzyme

In order to evaluate whether EP-4OCB-FMA and EP-4OCB-MA synthesized above were substrates of DPP-IV, first, they were reacted with a purified enzyme, and then the products were assayed by LC/MS.

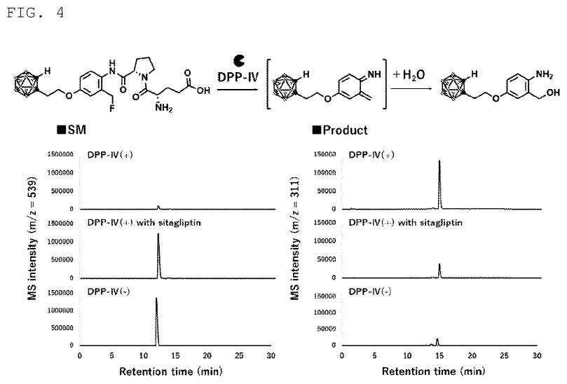

First, the result of EP-4OCB-FMA is shown below. EP-4OCB-FMA was completely consumed by addition of DPP-IV, and a benzyl alcohol form was confirmed as a product (FIG. 4). This is considered to be due to the reaction of the generated azaquinone methide intermediate with the water molecules in the buffer as nucleophiles, which indirectly indicates the generation of an azaquinone methide intermediate. In addition, since the enzymatic reaction was suppressed in the presence of sitagliptin as an inhibitor of DPP-IV, it was confirmed that EP-4OCB-FMA was served as a substrate of DPP-IV.

Here, FIG. 4 illustrates LC/MS assay of the enzymatic reaction of EP-4OCB-FMA (100 μM): the results of mass chromatogram of EP-4OCB-FMA and the product compound after incubation at 37° C. for 12 hours in the presence or absence of DPP-IV (>0.1 U/mL), in the presence or absence of sitagliptin (200 μM), and in PBS (pH 7.4) (1% DMSO as a co-solvent).

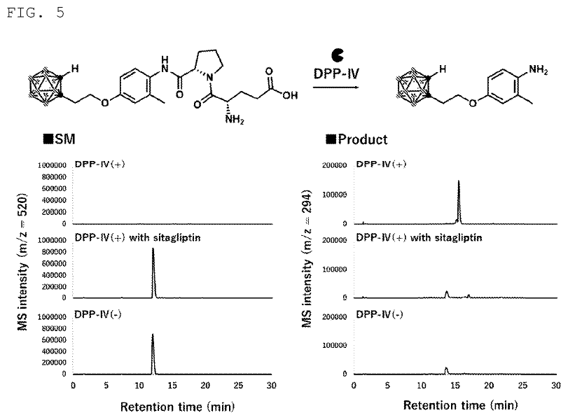

Similarly for EP-4OCB-MA, it was confirmed that a toluidine form was produced by addition of DPP-IV, and the enzymatic reaction was inhibited by addition of an inhibitor (FIG. 5).

Here, FIG. 5 illustrates LC/MS assay of the enzymatic reaction of EP-4OCB-MA (100 μM): the results of mass chromatogram of EP-4OCB-MA and the product compound after incubation at 37° C. for 12 hours in the presence or absence of DPP-IV (>0.1 U/mL), in the presence or absence of sitagliptin (200 μM), and in PBS (pH 7.4) (1% DMSO as a co-solvent).

### Example 3

### Evaluation of Cell Membrane Permeability, Cell Selectivity, and Cytotoxicity by CCK-8 Assay

Next, a CCK-8 assay using a DPP-IV high/low expression cell line was performed.

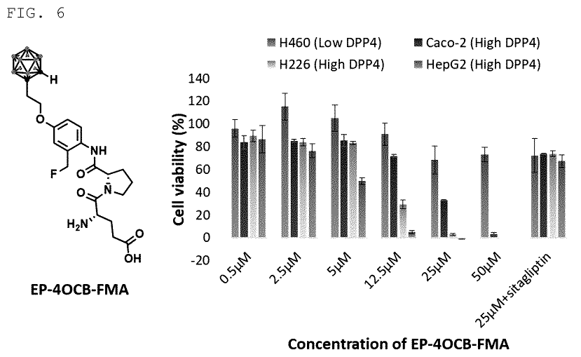

First, the result of EP-4OCB-FMA is shown below. When the cell viability was evaluated at 24 hours after the administration of EP-4OCB-FMA, a concentration-dependent decrease in cell viability was confirmed in H226 cells, HepG2 cells, and Caco-2 cells which were DPP-IV highly expressing strains. The results are illustrated in FIG. 6.

FIG. 6 illustrates the results of measuring cell viability in an EP-4OCB-FMA treatment for 24 hours in the presence or absence of CCK8 and sitagliptin. in which a mean value±a standard deviation of concentrations compared to an untreated case is illustrated (n=3 biological replicates).

On the other hand, it became clear that the cell viability of H460 cells, which were DPP-IV expressing strains, was hardly reduced even when the cells were administered at a high concentration of 50 μM. In addition, also in the DPP-IV highly expressing strain, the viability was significantly recovered by administration of sitagliptin as an inhibitor, and thus the result suggested that the cytotoxicity of EP-4OCB-FMA was dependent on DPP-IV. This is considered to be because the compound before the enzymatic reaction has low cell membrane permeability, but the hydrophobicity is improved after the enzymatic reaction, and the cell membrane permeability is improved.

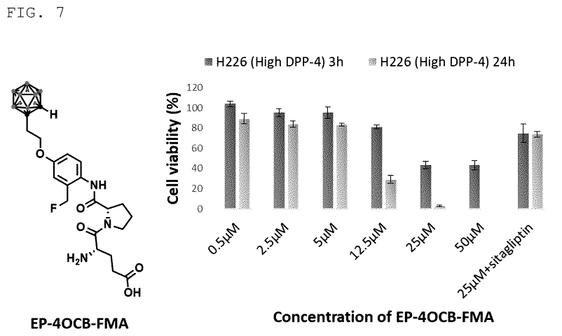

In addition, when the same evaluation was performed on H226 cells highly expressing DPP-IV 3 at hours after administration of EP-4OCB-FMA, a time-dependent decrease in cell viability was confirmed. The results are illustrated in FIG. 7.

FIG. 7 illustrates the results of measuring cell viability in an EP-4OCB-FMA treatment for 3 hours or 24 hours in the presence or absence of CCK8 and sitagliptin. in which a mean value±a standard deviation of concentrations compared to an untreated case is illustrated (n=3 biological replicates).

Since there is no difference in cell viability between the 25 μM administration group and the 50 μM administration group, it can be explained by considering that EP-4OCB-FMA is gradually metabolized by DPP-IV on the cell membrane and is still in the middle at the stage of 3 hours after administration.

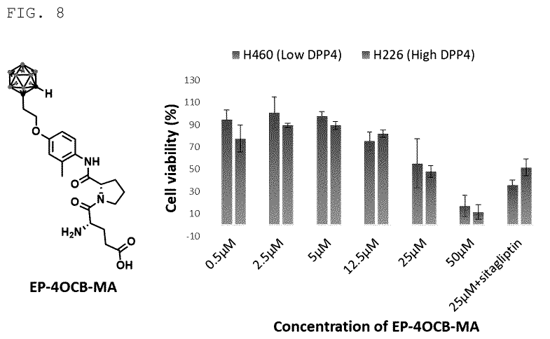

On the other hand, for EP-4OCB-MA as a control compound, a concentration-dependent decrease in cell viability was confirmed regardless of high expression/low expression of DPP-IV. The results are illustrated in FIG. 8.

FIG. 8 illustrates the results of measuring cell viability in an EP-4OCB-MA treatment for 24 hours in the presence or absence of CCK8 and sitagliptin. in which a mean value±a standard deviation of concentrations compared to an untreated case is illustrated (n=3 biological replicates).

Based on the fact that the cell viability was not recovered even by addition of the inhibitor sitagliptin, it was suggested that toxicity was exhibited independently of DPP-IV.

### Example 4

### Evaluation of Selectivity and Cellular Uptake in Cultured Cell Lines

When a degree of accumulation of a BNCT drug in a cell or a tumor tissue is evaluated, a boron quantitative method by an inorganic element analysis method such as inductively coupled plasma mass spectrometry (ICP-MS) or inductively coupled plasma atomic emission spectrometry (ICP-AES) is widely used in both in vitro and in vivo systems. Among them, a microwave nitrogen plasma atomic emission spectrometer (MP-AES) is an apparatus capable of safely and easily quantifying boron without using an expensive gas or a combustible gas.

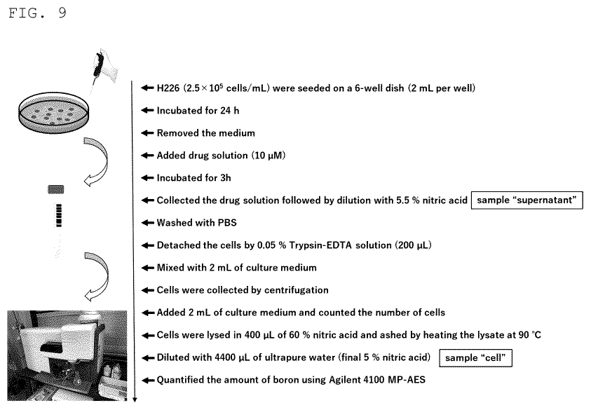

According to the above Examples, EP-4OCB-FMA was expected to selectively accumulate cells highly expressing DPP-IV due to a change in cell membrane permeability before and after the enzymatic reaction. In order to evaluate this in more detail, the intracellular boron concentration at 3 hours after the administration of EP-4OCB-FMA was quantified using MP-AES. The protocol used is illustrated in FIG. 9. From the results of the CCK-8 assay in H226 cells, it was considered that 3 hours after administration at 10 μM was appropriate as a condition under which the cells were not considered to kill, and the intracellular boron concentration was quantified.

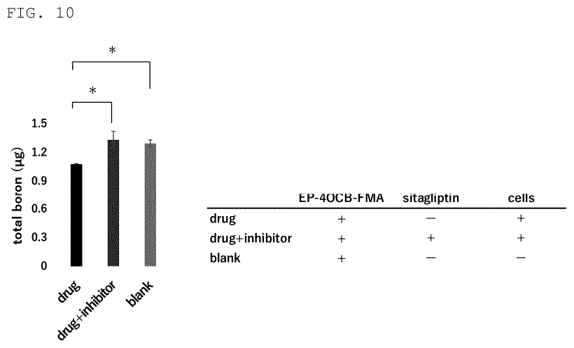

The results of the quantification of extracellular boron are illustrated in FIG. 10. H226 cells were cultured with EP-4OCB-FMA in the presence or absence of a DPP-IV inhibitor (sitagliptin) for 3 hours. For blank evaluation, cells were not seeded and only a medium containing a drug was added. The results are shown as a mean value±a standard deviation (n=3 biological replicates).

When the boron concentration in the extracellular fluid was first quantified at 3 hours after the administration of EP-4OCB-FMA, it was confirmed that the boron concentration in the drug administration group was lower than that in the blank group containing only the drug without cells. In addition, in the presence of sigatgliptin as a DPP-IV inhibitor, a decrease in the boron concentration in the extracellular fluid was hardly confirmed, and thus it was suggested that EP-4OCB-FMA was taken into cells only by an enzymatic reaction with DPP-IV on the cell membrane.

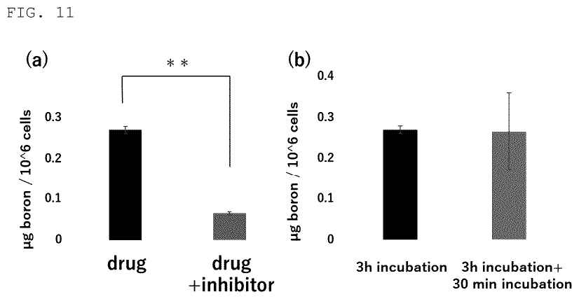

Furthermore, when the intracellular boron concentration was quantified, it became clear that EP-4OCB-FMA was accumulated in the cells at a high concentration of 0.27 μg of boron per 10⁶ cells in the H226 cells known to have high DPP-IV expression (FIG. 11). The test conditions and the like are as follows.

FIG. 11 (a): Cellular uptake of EP-4OCB-FMA H226 cells were cultured with EP-4OCB-FMA in the presence or absence of a DPP-IV inhibitor (sitagliptin) for 3 hours. The results are shown as a mean value±a standard deviation (n=3 biological replicates). **P<0.001 (Student's t-test)

FIG. 11(b): Intracellular retention of EP-4OCB-FMA Cells were cultured with EP-4OCB-FMA for 3 hours in the presence or absence of further culture in a fresh medium without EP-4OCB-FMA. The result of the case where an additional culture is not performed is the same as in (a). The results are shown as a mean value±a standard deviation (n=3 biological replicates).

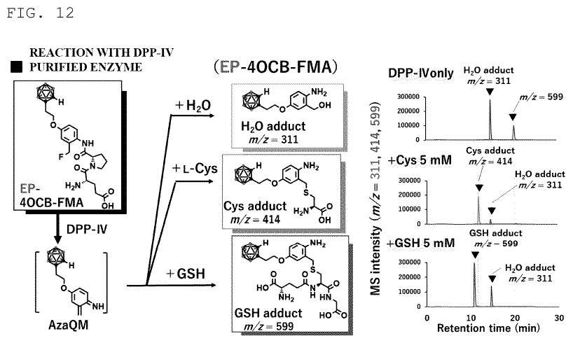

Furthermore, addition of the DPP-IV inhibitor sitagliptin reduced the intracellular boron concentration to about ¼, and DPP-IV-dependent intracellular accumulation became clear (see FIG. 12(a)). It is said that Tumor/Normal (T/N) as a guideline for performing a BNCT is 3, which suggests that EP-4OCB-FMA is promising as a BNCT drug.

In addition, when the medium containing the drug was removed, incubation for 30 minutes after replacement with a fresh medium was performed as a wash operation, and then the intracellular boron concentration was similarly quantified, the concentration was almost unchanged from before the wash operation (see FIG. 11(b)).

Thus, it was suggested that EP-4OCB-FMA had excellent intracellular retention.

### Example 5

### Evaluation of Reactivity Between Azaquinone Methide Intermediate and Nucleophilic Group

In the pharmaceutical strategy of EP-4OCB-FMA, an azaquinone methide intermediate generated after an enzymatic reaction with DPP-IV reacts with an intracellular nucleophile to acquire intracellular retention. As a study to confirm that this function works, an in vitro study using a purified enzyme was conducted. Specifically, EP-4OCB-FMA reacted with a DPP-IV purified enzyme in the presence of L-cysteine or glutathione, whether or not these nucleophiles reacted with an azaquinone methide intermediate was evaluated using LC/MS.

The protocol used was obtained by adding the same protocol as that used for confirming the reactivity of EP-4OCB-FMA of Example 2 with the purified enzyme to the following operation.
In an experiment for evaluating the reaction of an azaquinone methide with a nucleophilic reagent (GSH, 1-Cys), a 0.1 M HEPES buffer (pH 7.4) containing 5 mM of each nucleophile was prepared and used.

The results are shown below (FIG. 12). When the reaction product was incubated with the purified enzyme in the presence of 5 mM cysteine and assayed 12 hours later, an MS peak of a compound that appeared to be the nucleophilic addition of cysteine to the azaquinone methide intermediate was detected. In addition, since the MS peak of the water adduct detected at the same time was much lower in intensity than when cysteine was not added, it was suggested that water molecules and cysteine competed with each other and reacted with the azaquinone methide intermediate.

In addition, in the case where cysteine was not added, an MS peak with m/z=599 was detected, and from the value and the shape of the isotope peak derived from carborane, it was presumed that the MS peak was derived from the following compound, but this signal was hardly detected in the presence of cysteine.

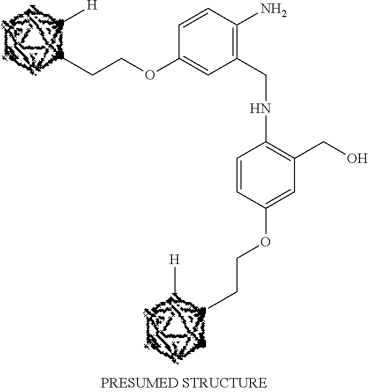

In the presence of 5 mM glutathione, the reactivity of the SH group of glutathione was lower than that of cysteine, and therefore more water adducts were formed than in the case of cysteine, but an MS signal that appeared to be a glutathione adduct was also detected. From the above, it became clear that the azaquinone methide intermediate reacted with cysteine or glutathione as an intracellular nucleophile to form a covalent bond, and it was suggested that the boron drug was trapped by the intracellular nucleophile as designed.

FIG. 12 illustrates LC/MS assay of the enzymatic reaction of EP-4OCB-FMA (100 μM): the results of mass chromatogram of EP-4OCB-FMA and the product compound after incubation at 37° C. for 12 hours in the presence or absence of DPP-IV (>0.1 U/mL) and in PBS (pH 7.4) (1% DMSO as a co-solvent).

### Synthesis Example 3

EP-4ACB-FMA (compound 36), as a compound of the present invention in which a carborane moiety and a benzene ring moiety were connected by an alkyl group, was synthesized according to the following Synthesis Scheme 4. Details of each reaction step will be described below.

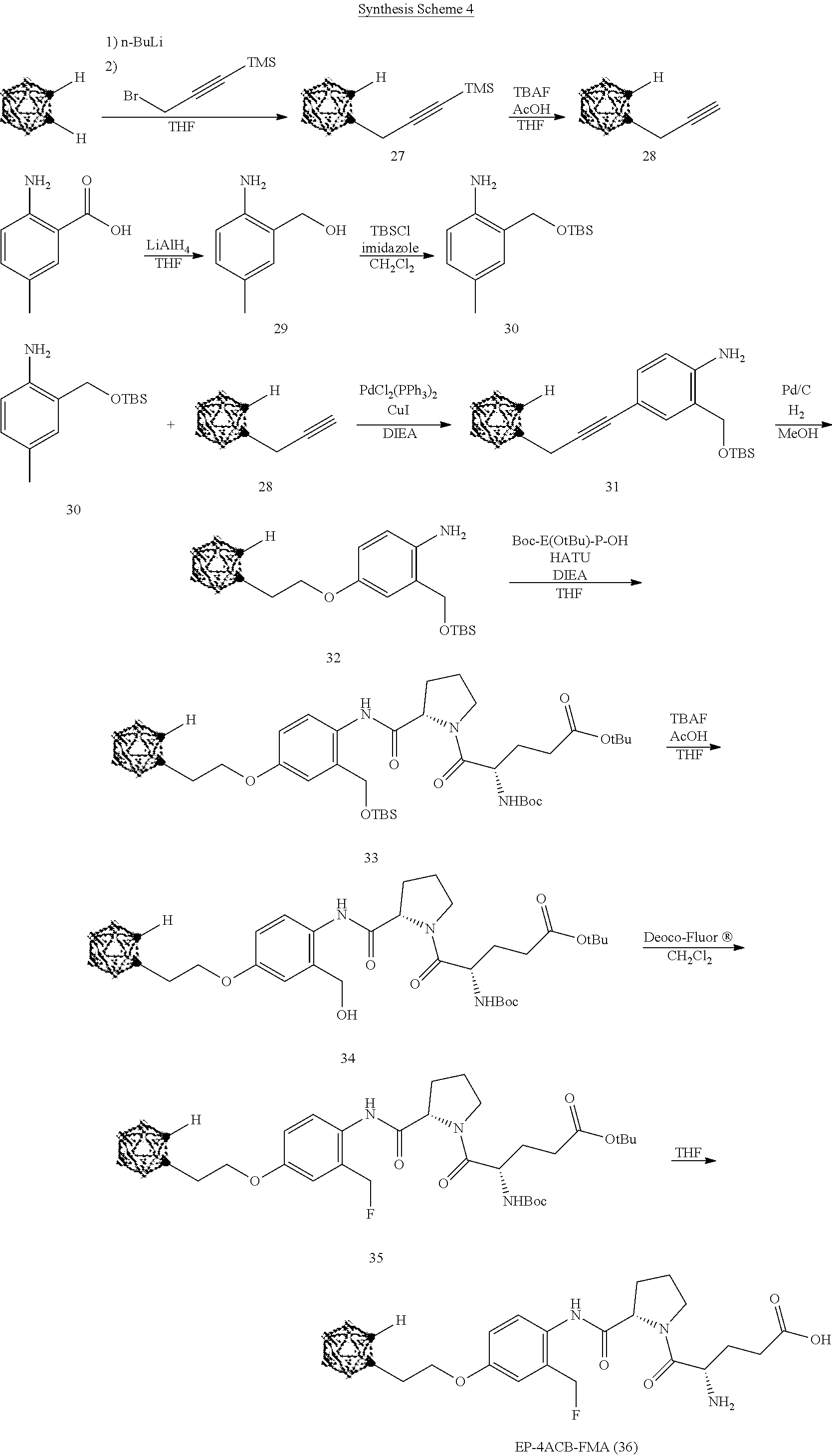

(1) Synthesis of Compound 27 ([3-(trimethylsilyl)prop-2-ynyl]-o-carborane)

o-Carborane (689 mg, 4.78 mmol) was dissolved in 15 mL of dry THF. After cooling to -20° C., a hexane solution of 1.6 M n-BuLi (3.6 mL, 5.8 mmol) was added dropwise to the solution. After stirring in an argon atmosphere at −20° C. for 30 minutes, 2 mL of a dry THE solution of 3-bromo-1-(trimethylsilyl)-1-propyne (1.0 g, 5.2 mmol) was added dropwise. After stirring at −20° C. , the reaction solution was warmed to room temperature. After stirring for 2.5 hours, the reaction was quenched with H₂O. The mixture was concentrated under low pressure. The residue was diluted with AcOEt and a saline solution, and then the organic layer was extracted, dried with Na₂SO₄, and concentrated under low pressure. The residue was purified by MPLC (OH silica gel, AcOEt/hexane) to obtain light yellow oily matter (888 mg, 3.49 mmol, 73%).

¹H-NMR (400 MHz, CDCl₃) δ 3.93 (s, 1H), 3.20 (s, 2H), 0.18 (s, 9H), 1.40-3.20 (br, 10H).
(2) Synthesis of Compound 28 (prop-2-ynyl-o-carborane)

The compound 27 (782 mg, 3.07 mmol) was dissolved in 7 mL of dry THF, and subsequently, TBAF (in a THE solution, about 1 M) (3.38 mL, 3.38 mmol) and acetic acid (229 μL, 4.00 mmol) were added. The solution was stirred in an argon atmosphere at room temperature for 1 hour. The solvent was removed by evaporation. AcOEt was added to the residue, and the organic layer was washed with a saline solution. The organic layer was dehydrated with Na₂SO₄ and concentrated under low pressure. The residue was purified by MPLC (OH silica gel, AcOEt/hexane) to obtain a transparent solid (393 mg, 2.16 mmol, 70%).

¹H-NMR (400 MHz, CDCl₃) δ 4.01 (s, 1H), 3.21 (d, J=2.7 Hz, 2H), 2.37 (t, J=2.7 Hz, 1H), 1.40-3.20 (br, 10H). ¹³C-NMR (101 MHz, CDCl₃) δ 76.35, 75.01, 69.65, 59.61, 28.31.

### (3) Synthesis of Compound 29

2-Amino-5-iodobenzoic acid (2.0 g, 4.35 mmol) was dissolved in 15 mL of dry THF, and subsequently, LiAlH₄ (2.5 M in a THE solution) (6 mL, 15 mmol) was added dropwise at 0° C. After heating to room temperature, the solution was stirred at room temperature for 4 hours. After carefully quenching the reaction with AcOEt, the solvent was diluted with H₂O. The mixture was concentrated to remove THF, and filtration was performed with Celite (registered trademark). After washing the precipitate with AcOEt, the filtrate was extracted with AcOEt, and then, the organic layer was dried with Na₂SO₄ and concentrated under low pressure. The residue was purified by MPLC (OH silica gel, AcOEt/hexane) to obtain a yellow powder (707 mg, 2.84 mmol, 38%).

### ¹H-NMR (400 MHz, MeOH-d4) δ 7.38 (d, J=2.3 Hz, 1H), 7.30 (dd, J=8.5, 2.3 Hz, 1H), 6.53 (d, J=8.5 Hz, 1H), 4.49 (s, 2H).

### (4) Synthesis of Compound 30

The compound 29 (601 mg, 2.41 mmol), TBSCl (591 mg, 3.92 mmol), and imidazole (399 mg, 5.86 mmol) were added to 10 mL of dry CH₂Cl₂. The solution was stirred in an argon atmosphere at room temperature for 2 hours. AcOEt was added to the solvent, and the organic layer was washed with water. The organic layer was dehydrated with Na₂SO₄ and concentrated under low pressure. The residue was purified by MPLC (OH silica gel, AcOEt/hexane) to obtain yellow oily matter (855 mg, 2.35 mmol, 98%).

¹H-NMR (400 MHz, CDCl₃) δ 7.34 (dd, J=8.5, 2.3 Hz, 1H), 7.31 (d, J=2.3 Hz, 1H), 6.44 (d, J=8.5 Hz, 1H), 4.60 (s, 2H), 4.22 (br, 2H), 0.89 (s, 9H), 0.07 (s, 6H).

### (5) Synthesis of Compound 31

The compound 30 (582 mg, 1.60 mmol) and the compound 20 (340 mg, 1.87 mmol) were dissolved in 5 mL of DIEA, and then, copper (I) iodide (33 mg, 0.17 mmol) was added. After degassing by a freeze-pump-thaw cycle, PdCl₂(PPh₃)₂ (56 mg, 0.080 mmol) was added to the solution. The reaction solution was stirred in an argon atmosphere at 70° C. for 6 hours. AcOEt was added to the solvent, and the organic layer was washed with water and a saline solution. The organic layer was dehydrated with Na₂SO₄ and concentrated under low pressure. The residue was purified by MPLC (OH silica gel, AcOEt/hexane) to obtain a yellow solid (370 mg, 0.877 mmol, 55%).

¹H-NMR (400 MHz, CDCl₃) δ 7.15 (dd, J=8.2, 1.8 Hz, 1H), 7.08 (d, J=1.8 Hz, 1H), 6.58 (d, J=8.2 Hz, 1H), 4.63 (s, 2H), 4.43 (s, 2H), 4.04 (s, 1H), 3.38 (s, 2H), 1.40-3.20 (br, 10H), 0.90 (s, 9H), 0.08 (s, 6H). ¹³C-NMR (101 MHz, CDCl₃) δ 147.27, 132.51, 131.95, 124.98, 115.46, 110.00, 87.28, 79.17, 71.23, 64.56, 59.58, 29.38, 25.92, 18.31, −5.17

### (6) Synthesis of Compound 32

The compound 31 (321 mg, 0.769 mmol) was dissolved in 10 mL of methanol, and subsequently, a small amount of Pd/C (10%, 55% water) was added. After stirring under H₂ at room temperature for 3 hours, the reaction mixture was filtered with Celite (registered trademark), and the filtrate was concentrated under low pressure. The residue was purified by MPLC (OH silica gel, AcOEt/hexane) to obtain yellow oily matter (321 mg, 0.761 mmol, 99%). ¹H-NMR (400 MHz, CDCl₃) δ 6.85 (dd, J=7.8, 1.8 Hz, 1H), 6.79 (d, J=2.3 Hz, 1H), 6.60 (d, J=8.2 Hz, 1H), 4.65 (s, 2H), 3.51 (s, 1H), 2.47 (t, J=7.3 Hz, 2H), 2.14-2.19 (m, 2H), 1.70-1.74 (m, 2H), 1.40-3.20 (br, 10H), 0.90 (s, 9H), 0.07 (s, 6H). ¹³C-NMR (101 MHz, CDCl₃) δ 144.47, 129.64, 128.45, 128.35, 125.54, 116.03, 75.38, 64.90, 61.19, 37.44, 34.11, 31.14, 25.97, 18.36, −5.11.

### (7) Synthesis of Compound 33

The compound 32 (139 mg, 0.330 mmol) and N-Boc-E(OtBu)—P—OH (171 mg, 0.430 mmol) were dissolved in 2 mL of dry THF, and subsequently, HATU (377 mg, 0.991 mmol) and DIEA (173 μL, 0.990 mmol) were added. The solution was stirred in an argon atmosphere at room temperature for 3 hours. The solvent was removed by evaporation. AcOEt was added to the residue, and the organic layer was washed with a saline solution. The organic layer was dried with Na₂SO₄ and concentrated under low pressure. The residue was purified by MPLC (OH silica gel, AcOEt/hexane) to obtain a white powder (209 mg, 0.260 mmol, 79%).

¹H-NMR (400 MHz, CDCl₃) δ 8.99 (s, 1H), 7.95 (d, J=8.2 Hz, 1H), 7.01 (dd, J=8.2, 1.8 Hz, 1H), 6.95 (d, J=1.8 Hz, 1H), 5.23 (d, J=8.7 Hz, 1H), 4.79 (d, HBn, J_{gem}=12.8 Hz, 1 H), 4.61 (d, HBn, J_{gem}=12.8 Hz, 1H), 4.51-4.56 (m, 2H), 3.80 (m, 2H), 3.52 (s, 1H), 2.55 (t, J=7.1 Hz, 2H), 2.30-2.42 (m, 2H), 2.00-2.28 (m, 6H), 1.69-1.80 (m, 4H), 1.44 (s, 9H), 1.43 (s, 9H), 1.40-3.20 (br, 10H), 0.92 (s, 9H), 0.12 (s, 3H), 0.07 (s, 3H). ¹³C-NMR (101 MHz, CDCl₃) δ 172.23, 171.81, 169.44, 155.69, 136.04, 135.12, 130.47, 128.12, 127.44, 122.34, 80.62, 79.76, 75.16, 64.34, 61.35, 61.07, 51.20, 47.44, 37.32, 34.38, 31.00, 30.76, 28.72, 28.42, 28.18, 28.01, 25.94, 25.25, 18.35, −5.07

### (8) Synthesis of Compound 34

The compound 33 (182 mg, 0.226 mmol) was dissolved in 3 mL of dry THF, and subsequently, TBAF (in a THE solution, about 1 M) (726 μL, 0.726 mmol) and acetic acid (27.7 μL, 0.484 mmol) were added. The solution was stirred in an argon atmosphere at room temperature for 40 minutes, and the solvent was removed by evaporation. AcOEt was added to the residue, and the organic layer was washed with water. The organic layer was dehydrated with Na₂SO₄ and concentrated under low pressure. The residue was purified by MPLC (OH silica gel, AcOEt/hexane) to obtain a white solid (132 mg, 0.191 mmol, 85%).

¹H-NMR (400 MHz, CDCl₃) δ 9.29 (s, 1H), 7.89 (d, J=8.2 Hz, 1H), 7.05 (dd, J=8.2, 1.8 Hz, 1H), 6.96 (d, J=1.8 Hz, 1H), 5.32 (d, J=8.7 Hz, 1H), 4.73 (dd, J=7.8, 2.7 Hz, 1H), 4.61 (s, 2H), 4.56 (td, J=8.7, 3.7 Hz, 1H), 3.87 (brs, 1H), 3.73-3.83 (m, 2H), 3.54 (s, 1H), 2.53 (t, J=7.3 Hz, 2H), 2.23-2.40 (m, 3H), 2.05-2.19 (m, 6H), 1.71-1.85 (m, 3H), 1.44 (s, 9H), 1.41 (s, 9H), 1.40-3.20 (br, 10H). ¹³C-NMR (101 MHz, CDCl₃) δ 173.25, 172.62, 169.69, 155.57, 136.72, 135.19, 130.78,

### (9) Synthesis of Compound 35

The compound 34 (112 mg, 0.162 mmol) was dissolved in 6 mL of dry CH₂Cl₂, and then a solution of Deoxo-Fluor (registered trademark) (59.7 μL, 0.324 mmol) was added dropwise to 4 mL of dry CH₂Cl₂ at 0° C. The reaction solution was stirred in an argon atmosphere at room temperature for 1 hour. CH₂Cl₂ was added to the reaction solution and washed with a NaHCO₃ saturated aqueous solution. The organic layer was dehydrated with Na₂SO₄ and concentrated under low pressure. The residue was purified by MPLC (OH silica gel, AcOEt/hexane) to obtain a transparent solid (88 mg, 0.127 mmol, 78%).

¹H-NMR (400 MHz, CDCl₃) δ 8.95 (s, 1H), 7.83 (d, J=8.7 Hz, 1H), 7.13 (d, J=8.2 Hz, 1H), 7.08 (s, 1H), 5.30-5.42 (m, 3 H), 4.78 (dd, J=8.0, 2.1 Hz, 1H), 4.55 (td, J=9.4, 3.5 Hz, 1H), 3.75 (dd, J=7.8, 5.0 Hz, 2H), 3.54 (s, 1H), 2.48-2.58 (m, 3H), 2.28-2.42 (m, 2H), 2.16-2.20 (m, 2H), 1.94-2.11 (m, 3H), 1.71-1.81 (m, 4H), 1.44 (s, 9H), 1.43 (s, 9H), 1.40-3.20 (br, 10H). ¹³C-NMR (101 MHz, CDCl₃) δ 173.33, 172.19, 169.37, 155.64, 137.15, 134.75, 134.72, 129.83, 129.80, 129.30, 129.24, 127.37, 127.21, 123.68, 83.32, 81.68, 80.83, 79.97, 77.46, 77.15, 76.83, 75.03, 61.36, 60.84, 51.18, 47.67, 37.43, 34.35, 31.05, 30.76, 28.41, 28.14, 28.06, 27.18, 25.31.

### (10) Synthesis of Compound 36

The compound 35 (53 mg, 0.077 mmol) was dissolved in 1 mL of TFA. The solution was stirred at room temperature for 10 minutes, and the reaction solution was diluted with CH₂Cl₂ and concentrated by evaporation. The residue was purified by HPLC (eluent: A: H₂O, 0.1% TFA (v/v), eluent: B: CH₃CN/H₂O=80/20, 0.1% TFA (v/v)) to obtain a white powder (24 mg, 0.045 mmol, 58%).

¹H-NMR (400 MHz, MeOH-D4) δ 7.19-7.30 (m, 3H), 5.24-5.49 (m, 2H), 4.63 (dd, J=8.2, 5.5 Hz, 1H), 4.52 (s, 1H), 4.38 (dd, J=7.4, 4.9 Hz, 1H), 3.68-3.80 (m, 2H), 2.55-2.64 (m, 4H), 1.98-2.42 (m, 8H), 1.76-1.84 (m, 2H), 1.40-3.20 (br, 10H). ¹³C-NMR (101 MHz, Acetonitrile-d3) δ 175.17, 170.52, 167.60, 139.29, 133.30, 133.26, 130.64, 130.48, 129.36, 128.84, 128.77, 125.27, 82.56, 80.96, 76.29, 62.37, 61.00, 51.47, 47.50, 36.79, 33.85, 30.67, 29.00, 24.96, 24.88

1. A compound represented by the following General Formula (I) or a salt thereof. 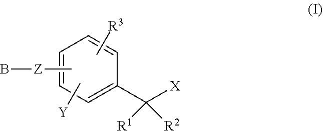
in the formula,
X is selected from the group consisting of a fluorine atom, an ester group (—OC(═O)—R′), a carbonate group (—OCO₂—R′), a carbamate group (—OCONH—R′), phosphoric acid and an ester group thereof (—OP(═O)(—OR′)(—OR″)), and sulfuric acid and an ester group thereof (—OSO₂—OR′),
where R′ and R″ are each independently selected from a substituted or unsubstituted alkyl group or a substituted or unsubstituted aryl group;
Y is —NH—CO-L, —NH-L′, or —OL′,
where L is a partial structure of an amino acid,
L′is a saccharide or a partial structure of a saccharide, a saccharide having a self-cleaving linker, or an amino acid or a peptide having a self-cleaving linker;
R¹ and R² are each independently selected from a hydrogen atom or a monovalent substituent;
R³ is a hydrogen atom or one to three identical or different monovalent substituents present on a benzene ring;
Z represents a single bond or a linking group; and
B represents a group containing ¹⁰B.

2. The compound or a salt thereof according to claim 1, wherein B is a group derived from a compound having at least one boron atom in a molecule.

3. The compound or a salt thereof according to claim 1, wherein B is a group derived from a boron cluster.

4. The compound or a salt thereof according to claim 3, wherein the boron cluster has a polyhedral structure.

5. The compound or a salt thereof according to claim 1, wherein B is a group derived from closo-dodecaborate, closo-carborane, nidocarborane, a bisdicarbolide metal complex, GB10, 1,2-dicarbacloso-dodecarborane, 1,7-dicarba-closo-dodecarborane, 1,12-dicarba-closo-dodecarborane, dicarba-closo-decarborane, or sulfur-substituted undecahydrododecaborate.

6. The compound or a salt thereof according to claim 1, wherein the linking group is selected from the group consisting of an alkylene group (where one or more —CH₂— of the alkylene group may be replaced by —O—, —S—, —NH—, or —CO—), arylene (including heteroarylene), cycloalkylene, an alkoxyl group, a polyethylene glycol chain, and a group constituted by optionally binding two or more groups selected from these groups.

7. The compound or a salt thereof according to claim 1, wherein the partial structure of the amino acid of L constitutes an amino acid, an amino acid residue, a peptide, or a part of an amino acid together with C═O to which L is bonded.

8. The compound or a salt thereof according to claim 1, wherein the partial structure of the saccharide of L′ constitutes a saccharide or a part of a saccharide together with O to which L′is bonded.

9. The compound or a salt thereof according to claim 1, wherein —Y in General Formula (I) is bonded to —C(R¹)(R²)X at an ortho position or a para position of a benzene ring.

10. The compound or a salt thereof according to claim 1, wherein Y has a structure selected from the following:

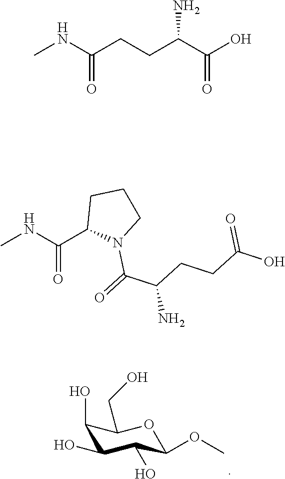

11. The compound or a salt thereof according to claim 1, wherein X is a fluorine atom or an ester group (—OC(═O)—R′).

12. The compound or a salt thereof according to claim 1, wherein R¹ and R² are each independently selected from a hydrogen atom or a fluorine atom.

13. The compound or a salt thereof according to claim 1, wherein the monovalent substituent of R³ is selected from the group consisting of an alkyl group, an alkoxycarbonyl group (—C(═O)—OR′), a nitro group, an amino group, a hydroxyl group, an alkylamino group (—NHR′ or —NR′₂—NHCOR′), an alkoxy group (—OR′), an ester group (—O—CO—R′), an amide group (—NHCOR′), a halogen atom, a boryl group, and a cyano group, where R′ is a substituted or unsubstituted alkyl group or a substituted or unsubstituted aryl group, and when there are two or more R's, R's may be the same as or different from each other.

14. The compound or a salt thereof according to claim 13, wherein the monovalent substituent of R³ is an alkyl group or an alkoxy group.

15. The compound or a salt thereof according to claim 13, wherein the monovalent substituent of R³ is a halogen atom.

16. The compound or a salt thereof according to claim 13, wherein one or more of the monovalent substituents of R³ are alkyl groups or alkoxy groups, and one or more of the monovalent substituents of R³ are halogen atoms.

17. The compound or a salt thereof according to claim 1, wherein all of R³s are hydrogen atoms.

18. A pharmaceutical composition comprising the compound according to claim 1 or a pharmaceutically acceptable salt thereof.

19. The pharmaceutical composition according to claim 18, wherein the pharmaceutical composition is used in a boron neutron capture therapy.

20. The pharmaceutical composition according to claim 19, wherein the pharmaceutical composition is accumulated in cancer cells by acting selectively on cells by a cancer cell-specific enzyme activity.

21. The pharmaceutical composition according to claim 20, wherein the enzyme is a peptidase or a glycosidase.

22. A method for diagnosing, treating, or diagnosing and treating a disease or a symptom that may cause the disease, the method comprising:
(A) administering, to a subject having or suspected of having a disease or a symptom, a pharmaceutical composition comprising the compound according claim 1 or a pharmaceutically acceptable salt thereof; and
(B) irradiating a ¹⁰B atom localized in a target tissue of the subject with a neutron beam to perform a boron neutron capture therapy on the target tissue.
